(19) United States
(12) Patent Application Publication
(10) Pub. No.: US 2026/0274931 A1
(43) Pub. Date: Sep. 17, 2026

(54) METHODS OF TREATING ALZHEIMER'S DISEASE

(71) Applicant: KLARITOS, INC, Menlo Park, CA (US)

(72) Inventor: Vijay RAMAKRISHNAN, Palo Alto, CA (US)

(21) Appl. No.: 19/168,282

(22) PCT Filed: Mar. 22, 2024
(86) PCT No.: PCT/US2024/021163

§ 371 Date: Sep. 23, 2025

Related U.S. Application Data
(60) Provisional application No. 63/454,124, filed on Mar. 23, 2023.

(51) Int. Cl. C07K 16/18; A61K 38/17; A61K 39/00; A61K 39/395; A61P 25/28; C07K 16/24; C07K 16/28; C12Q 1/6883; G01N 33/50; G01N 33/68; G16H 10/20; G16H 10/60

(52) U.S. Cl. CPC C07K 16/18; A61K 38/1793; A61K 39/3955; A61P 25/28; C07K 16/241; C07K 16/2887; C12Q 1/6883; G01N 33/5005; G01N 33/6863; G01N 33/6869; G01N 33/6896; G16H 10/20; G16H 10/60; A61K 2039/505; A61K 2039/507; C07K 2317/21; C07K 2317/24; C07K 2317/732; C12Q 2600/112; C12Q 2600/156; G01N 2333/4709; G01N 2333/525; G01N 2333/5412; G01N 2333/545; G01N 2800/2821; G01N 2800/52; G01N 2800/56

(57) ABSTRACT

Disclosed herein are methods of conducting clinical trials in Alzheimer's disease (AD). Methods of selecting a medicament and treating an AD subject are disclosed. Methods, systems, kits, and devices for selecting a subject in AD clinical trials and treatment protocols are disclosed herein.

### CROSS-REFERENCE

This application claims the benefit of U.S. Provisional Application No. 63/454,124, filed Mar. 23, 2023, which is incorporated by reference herein in its entirety.

### SUMMARY

Disclosed are methods of conducting clinical trials and treating Alzheimer's Disease (AD) employing monotherapy or combination therapy approaches. The methods comprise stratifying AD subjects based on key therapeutic mechanisms of action of drugs, disease subtypes, or immune mechanisms to guide patient enrollment, clinical trials, and treatment decisions. In one embodiment, these strategies are designed to develop patient-centric, tailored therapies and protocols with an intent to significantly slow, halt, or reverse disease progression, or to achieve clinical remission. In another embodiment, this approach could achieve optimal clinical and financial outcomes for both patients and healthcare systems.

Disclosed herein are methods of conducting a clinical trial in AD comprising administering a monotherapy or a combination therapy to subjects enrolled in the trial. In one embodiment, the trial is conducted by stratifying AD subjects based on the therapeutic mechanism(s) of a drug and/or disease subtypes to make patient enrollment and clinical trial design decisions. In another embodiment, the trial comprises administering an anti-TNF therapy or a B-cell depleting antibody therapy or administering a combination therapy consisting of an anti-beta-amyloid (AB) antibody and either an anti-TNF therapy or a B-cell depleting therapy to select subsets of patients. In one embodiment, the trial is conducted to develop precision medicine therapies and protocols to slow, halt, or reverse disease progression, or to achieve clinical remission, in stratified subsets of patients or on a patient-by-patient basis.

Disclosed are methods of treating an AD patient with a monotherapy or a combination therapy. In one embodiment, the treatment protocol is conducted by stratifying AD subjects based on therapeutic mechanism(s) of a drug and/or disease subtypes to make treatment decisions in subsets of patients or on a patient-by-patient basis. In another embodiment, the trial comprises administering an anti-TNF therapy or a B-cell depleting antibody therapy or administering a combination therapy consisting of an anti-Aβ antibody and either an anti-TNF therapy or a B-cell depleting therapy in stratified subsets of patients. In yet another embodiment, the protocol provides precision medicine therapies to slow, halt, or reverse disease progression, or to achieve clinical remission, in a treated patient. In some embodiments, the treatment and payment decisions are made in stratified subsets on a patient-by-patient basis to achieve significantly better clinical and/or financial outcomes for patients and payers.

Methods are disclosed that comprise administering a monotherapy or a combination therapy to an AD subject enrolled in a clinical trial. This comprises: the monotherapy being an anti-Aβ antibody, lecanemab; the monotherapy being an anti-TNF therapy, adalimumab; and the combination therapy comprising lecanemab and adalimumab. In one embodiment, a clinical trial can be a Phase-2, Phase-3, or Phase-4 clinical trial. In another embodiment, a clinical trial can be a real-world evidence (RWE) trial, wherein all treated patients or subsets thereof are in the trial; the trial size is ultra-large, for instance, comprising 100,000 or more patients; and patients are stratified into subset(s) or on a patient-by-patient basis, for instance, based on the mechanism of action of drugs, mechanism of disease severity or progression, or by disease subtypes by measuring critical clinical parameters.

In one embodiment, the anti-Aβ antibody is lecanemab, donanemab, aducanumab, or gantenerumab. In another embodiment, the anti-TNF therapy can be an adalimumab biosimilar approved by a regulatory agency, and it is commercially available in the respective market(s). In another embodiment, the anti-TNF therapy can be etanercept. In yet another embodiment, a B-cell depleting therapy, for instance, an anti-CD20 antibody therapy such as rituximab can be a monotherapy; and the combination therapy can comprise lecanemab and rituximab. In another embodiment, the combination therapy can comprise adalimumab and rituximab.

In some embodiments, a regulatory agency can be the Food and Drug Administration (FDA). In another embodiment, the adalimumab biosimilar can be an FDA-approved biosimilar for treating an autoimmune disease, for instance, rheumatoid arthritis, and that it is not approved for treating AD and is not commercially available in the U.S. whereas it is approved by the European Medicines Agency (EMA) and commercially available in Europe for treating an autoimmune disease.

In some embodiments, patients recruited in an AD clinical trial have an underlying additional autoimmune disease or an inflammatory disease, wherein the disease is rheumatoid arthritis, polyarticular juvenile idiopathic arthritis, Crohn's disease, ulcerative colitis, ankylosing spondylitis, psoriasis, plaque psoriasis, or psoriatic arthritis, and wherein the subjects recruited in the trial are cognitively unimpaired (CU) elderly patients or mild cognitively impaired (MCI) patients. In another embodiment, a subset of CU patients exhibits subjective cognitive decline (SCD).

In one embodiment, disclosed is a method of conducting a clinical trial in AD subjects with an underlying autoimmune disease or inflammatory disease, comprising: stratifying patients based on AD pathology, underlying autoimmune or inflammatory disease, and biomarkers indicative of disease subtypes; administering a drug candidate either as a monotherapy or part of a combination therapy, wherein the monotherapy or combination therapy comprises at least one of the following: an anti-Aβ antibody, an anti-TNF biologic, or a B-cell depleting antibody; and assessing the dual effectiveness of the drug candidates on AD progression and the underlying condition through predefined clinical endpoints and biomarker changes. In an embodiment, the underlying autoimmune or inflammatory disease is selected from the group consisting of rheumatoid arthritis, psoriatic arthritis, Crohn's disease, ulcerative colitis, and inflammatory bowel disease, wherein the rheumatoid arthritis can be early or very early rheumatoid arthritis. In another embodiment, the AD pathology is selected from the group consisting of cognitively unimpaired (CU), subjective cognitive decline (SCD), mild cognitive impaired (MCI), or AD dementia states. In one embodiment, the clinical trial is a Phase-4 clinical trial. In another embodiment, it is a Phase-2b clinical trial.

In one embodiment, an AD trial is conducted in stratified subsets of AD patients, with subjects recruited on a subject-by-subject basis. The stratification is based on a key mechanism of action of a drug that determines its therapeutic efficacy. In an embodiment, a key mechanism is antibody-dependent cellular phagocytosis (ADCP). In yet another embodiment, a key mechanism is antibody-dependent cellular cytotoxicity (ADCC).

In one embodiment, stratification may be based on the disease subtypes of AD. This could include measuring markers such as inflammatory macrophages, B-cells, or subsets of B-cells on a subject-by-subject basis, as well as proinflammatory cytokines such as TNF-α, IL-1, IL-6, etc. In another embodiment, stratification may be based on the immune mechanisms such as amyloid-related imaging abnormalities (ARIA) and/or cerebral amyloid angiopathy (CAA), wherein the stratification includes functional polymorphisms in FcGR-3A, -2A receptors.

In one embodiment, the stratification methods can include conducting diagnostic tests to measure biomarkers from plasma or cerebrospinal fluid (CSF). These biomarkers may include the Aβ₄₂/Aβ₄₀ ratio, phosphorylated-tau217 (P-tau217), or neurofilament light chain (NfL). In one embodiment, diagnostic tests can vary, ranging from in vitro diagnostic tests, MRI or PET imaging to theragnostic tests. In another embodiment, the tests might evaluate the therapeutic and/or clinical efficacy of a monotherapy or a combination therapy. In certain embodiments, a diagnostic test might not require regulatory clearance from a regulatory agency. In yet another embodiment, diagnostic tests can include next-generation sequencing or accessing a database or data warehouse.

Also disclosed herein are methods that include administering a monotherapy or a combination therapy to an AD patient in a treatment protocol, wherein: the monotherapy can be an anti-Aβ antibody, lecanemab; optionally, the monotherapy can be an anti-TNF therapy, adalimumab; and the combination therapy comprises lecanemab and adalimumab. In some embodiments, a treatment protocol is a real-world evidence (RWE) trial, where all treated patients are included in the RWE trial; alternatively, it is a treatment-cum-trial protocol wherein clinical trial grade data is collected in every treated patient. In one embodiment, patients are stratified into subsets based on the primary mechanism of action of drugs or disease subtype(s). In another embodiment, the treatment protocol Is conducted on a subject-by-subject basis.

In an embodiment, the RWE trial is conducted by a third-party trial sponsor and the drug costs and/or trial expenses are covered by a third-party payer and not by the drug manufacturer. In another embodiment, the anti-Aβ antibody includes lecanemab, donanemab, aducanumab, or gantenerumab. In some embodiments, the anti-TNF therapy includes an adalimumab biosimilar that is approved by a regulatory agency and is commercially available in the respective market(s). Additionally, the anti-TNF therapy can include etanercept. In yet another embodiment, the B-cell depleting therapy, such as an anti-CD20 antibody like rituximab, serves as the monotherapy; and the combination therapy includes lecanemab and rituximab.

In some embodiments, a regulatory agency is the Food and Drug Administration (FDA). The adalimumab biosimilar may be an FDA-approved biosimilar for treating an autoimmune disease but is not approved for treating AD. In one embodiment, a CU individual with SCD who is selected for the treatment protocol may have an underlying additional autoimmune or an inflammatory disease, which includes rheumatoid arthritis, polyarticular juvenile idiopathic arthritis, Crohn's disease, ulcerative colitis, ankylosing spondylitis, psoriasis, plaque psoriasis, or psoriatic arthritis. In another embodiment, the AD patient presents with MCI.

In some embodiments, an AD treatment protocol stratifies patients into subsets, where the stratification is based on a key mechanism of action of a drug. In one embodiment, the key mechanism of action includes antibody-dependent cellular phagocytosis (ADCP). In another embodiment, the key mechanism is antibody-dependent cellular cytotoxicity (ADCC).

Stratification may also be based on subtypes of AD and the incidence of ARIA and CAA mechanisms. In some embodiments, it can be assessed by determining the FcGR-3A, -2A functional polymorphisms. In other embodiments, inflammatory macrophages, B-cells, immune complexes, subsets of B-cells, or the levels of cytokines such as TNF-α, IL-1, IL-6 in peripheral blood can be measured in stratified subsets or on a patient-by-patient basis.

The stratification method may include conducting a diagnostic test to measure biomarkers from plasma or CSF, where biomarkers include Aβ₄₂/Aβ₄₀ ratio, P-tau217 (including various forms of phosphorylated tau), or NfL. In one embodiment, diagnostic tests can range from in vitro diagnostic tests to MRI or PET imaging. In another embodiment, the tests might be theragnostic tests. In another embodiment, the tests can assess therapeutic and/or clinical efficacy. In some embodiments, a diagnostic test may not be approved or cleared by a regulatory agency. In another embodiment, diagnostic tests can include next-generation sequencing or evaluating plasma or CSF biomarkers of amyloid, tau, and neurodegeneration (ATN) in CU or CU-SCD individuals, or in MCI patients. In one embodiment, a diagnostic test may include accessing a database or data warehouse.

Also disclosed herein are methods of stratifying AD patients by measuring plasma biomarkers. In one embodiment, the Aβ₄₂/Aβ₄₀ ratio in plasma can be measured. In some embodiments, plasma phosphorylated-tau217 is utilized to differentiate AD from other neurogenerative diseases. In another embodiment, P-tau217 levels are utilized to detect early, preclinical stages of AD when insoluble tau aggregates cannot be detected yet by tau-PET imaging. In another embodiment, NfL can be measured in plasma. In other embodiments, all three biomarkers (ATN) are measured to improve prediction of cognitive decline in CU or CU-SCD populations, or in MCI patients. In an embodiment, an AD patient is beta-amyloid positive (Aβ⁺), tau negative (T⁻), and NfL negative (N⁻). In another embodiment, an AD patient is A⁺T⁺N⁻, while in some embodiments, an AD patient can be A⁺T⁺N⁺. In another embodiment, patients can be further stratified along the AD spectrum by cognitive staging, such as preclinical AD (CU, SCD), mild cognitive impairment (MCI), and AD dementia.

Also disclosed herein are methods of slowing, halting, or reversing the progression of AD in a subject enrolled in a monotherapy or combination therapy clinical trial that include: assigning CU, CU-SCD, or MCI patients diagnosed with an autoimmune disease, such as rheumatoid arthritis into a subject pool subset based on a selection from the group consisting of: immunoglobulin G (IgG) rheumatoid factor (RF)^{+/-}, IgG Anti-Citrullinated Protein Antibodies (ACPA)^{+/-}, Immunoglobulin A (IgA) RF^{+/-}, IgA ACPA⁺, C-Reactive Protein (CRP)^{Hi/Lo}, citrullinated fibrinogen (cFib)^{+/-}; and functional polymorphisms in Fc gamma receptors: FcGR-3A V/F¹⁵⁸, FcGR-2A H/R¹³¹, and FcGR-3B NA1/NA2. In one embodiment, the methods include: administering lecanemab as a first therapy to the subject, where this therapy is licensed and approved to treat MCI or early AD patients; administering adalimumab as a second therapy to the subject in the same clinical trial, where this therapy is licensed and approved to treat RA but not AD; wherein subjects in the combination therapy remain cognitively unimpaired for 1-3 or more years and achieve disease remission in RA after a time period of about six months. In an alternative embodiment, the trial may include CU or CU-SCD individuals, or MCI patients with no additional underlying autoimmune diseases.

In some embodiments, the information generated in an AD clinical trial or AD treatment protocol can be stored in a database, for example, an electronic database. Any method as described herein may utilize a database as a research tool, for use in a clinical trial or precision medicine treatment protocol. In some embodiment, a database can be a relational database. In some embodiments, the generated information can be stored in a data warehouse.

In some embodiments, the information may include details pertaining to conventional treatment, for example, electronic health records (EHR), claims data, payer and payment details, provider details, and may also include critical clinical parameters to determine, for instance, excellent, moderate, and poor responders to a treatment, disease remission and relapse details related to therapy, theragnostic details, staging of the disease, grading of the disease, subject-specific longitudinal treatment outcomes to a therapy, subject-specific disease progression and/or disease severity patterns, stratified subset-specific longitudinal treatment outcomes, stratified subset-specific disease progression and/or disease severity patterns, mechanistically correlated biomarkers, progression-free survival (PFS), progression patterns of AD pathology in stratified subsets, depletion and repopulation of inflammatory macrophages or subsets of B-cells, increase or decrease of the levels of cytokines such as TNF-α, IL-1, IL-6, in peripheral blood, and the like. In one embodiment, all such data combined, such as real-world data (RWD) and critical clinical parameters obtained from a conventional treatment platform, can be utilized to generate regulatory-grade clinical data.

In some embodiments, theragnostic evaluations are employed to generate critical clinical parameters. In one embodiment, disease and therapy management protocols are employed to develop critical clinical parameters. The information generated can be duly submitted to Federal agencies, for example, CMS, AHRQ, and FDA (or their foreign counterparts) on a timely basis. This information can also be submitted to the FDA for label revision, for example, label restriction, label expansion. In another embodiment, the information can be employed to develop synthetic control arms or digital twins.

In some embodiments, the information developed can be utilized to develop and provide patient-centric (N=1) assurances, such as efficacy and/or financial assurances in AD. Additionally, all these data can be stored in a data warehouse or system. In one embodiment, diagnostic tests may include plasma biomarkers of amyloid, tau, and neurodegeneration (ATN signature) in CU individuals or MCI patients. The information may include details from a clinical trial, such as an AD trial conducted in rheumatoid arthritis patients. Disclosed are methods of treating an AD patient by determining a category of therapeutic responsiveness to an anti-Aβ antibody therapy, like lecanemab therapy, by genotyping the individual for an FcGR-2A HH¹³¹ polymorphism and an FcGR-3A VV¹⁵⁸ polymorphism, where the FcGR-2A polymorphism and the FcGR-3A polymorphism together indicate a high degree of therapeutic responsiveness. The nine subsets of patients generated by the functional polymorphisms in FcGRs differ in ADCC and ADCP functions, which could be determined by in vitro or in vivo methods, or by ex vivo phagocytosis; and administering an effective amount of the antibody therapies, such as lecanemab and adalimumab, donanemab and rituximab, or adalimumab and rituximab to the individual. In one embodiment, this stratification protocol is utilized to recruit AD patients in a clinical trial, to recruit a subset of AD patients in a clinical trial, to select an AD patient for lecanemab treatment, to select a subset of AD patients for lecanemab treatment, to select a subset of AD patients for adalimumab treatment, to select an AD patient or a subset of AD patients for etanercept treatment, and to recruit a patient or a subset of AD patients in a rituximab clinical trial or a combination therapy trial involving lecanemab and rituximab or adalimumab and rituximab. In another embodiment, this stratification protocol is employed to treat a patient or a subset of AD patients with a rituximab monotherapy or a combination therapy involving lecanemab and rituximab or a combination therapy comprising adalimumab and rituximab.

Methods are provided for determining the degree of responsiveness to an antibody therapy by genotyping an AD subject for two or more FcGR functional polymorphisms and using the first and second FcGR polymorphisms to determine the subject's degree of responsiveness to antibody therapies, such as lecanemab, adalimumab, and rituximab, for example, Group-I as characterized by FcGR-3A VV¹⁵⁸ and FcGR-2A HH¹³¹, and administering one of these therapies to a subject in Group-I. Lecanemab employs ADCP as a key therapeutic mechanism whereas rituximab and adalimumab employ ADCC as a key therapeutic mechanism. In one embodiment, methods are provided for assessing the degree of responsiveness to an antibody or biologic therapy by genotyping the AD subject for two or more FcGR functional polymorphisms and utilizing the first and second FcGR polymorphisms to determine the subject's degree of responsiveness, such as etanercept, which does not employ ADCC as a key mechanism of action, for example, in Group-IX as defined by FcGR-3A FF¹⁵⁸ and FcGR-2A RR¹³¹, and administering etanercept to a subject in Group-IX.

Disclosed are methods of enrolling a patient in a clinical trial or for selecting a therapy for treatment of an ADCP- or ADCC-treatable disease by genotyping the subjects for an FcGR-2A functional polymorphism to classify the patient population into three groups (for example, HH¹³¹, HR¹³¹, RR¹³¹); genotyping the subjects for an FcGR-3 A functional polymorphism to classify the patient population into three groups (for example, VV¹⁵⁸, VF¹⁵⁸, FF¹⁵⁸); and classifying the subjects into nine patient groups I-IX based on the first and second results, and employing the first and second results to select a biologic, such as an antibody therapy that uses ADCC or ADCP as a key mechanism, to significantly improve the degree of treatment response in the patient group. In another embodiment, all or majority of patients in that patient group achieve significantly better treatment response. In one embodiment, subjects may be classified into nine groups: VV¹⁵⁸, HH¹³¹ (Group-1); VF¹⁵⁸, HH¹³¹ (Group-2); FF¹⁵⁸, HH¹³¹ (Group-3); VV¹⁵⁸, HR¹³¹ (Group-4); VF¹⁵⁸, HR¹³¹ (Group-5); FF¹⁵⁸, HR¹³¹ (Group-6); VV¹⁵⁸ RR¹³¹ (Group-7); VF¹⁵⁸, RR¹³¹ (Group-8); and FF¹⁵⁸, RR¹³¹ (Group-9). In another embodiment, AD is characterized as an ADCP-treatable disease treated with lecanemab or donanemab. RA can be characterized as an ADCC-treatable disease treated with adalimumab or rituximab. RA is also treated with etanercept that does not use ADCC as a key mechanism of action. In yet another embodiment, patients are further stratified based on the plasma levels of ATN biomarkers. In some embodiments, patients in Group-1 with A⁺T⁻N⁻ signature may be recruited in a clinical trial. Patients in Group-1 with an A⁺T⁺N⁻ signature may be recruited in a clinical trial. Patients in Group-4 with an A⁺T⁺N⁺ signature may be recruited in a clinical trial. Patients in Group-9 with an A⁺T⁺N⁻ signature may be recruited in a clinical trial.

Also disclosed herein are methods that include administering a monotherapy or a combination therapy to an AD subject selected from a stratified patient population in a clinical trial, wherein: the monotherapy can be an anti-Aβ antibody, lecanemab; or an anti-TNF therapy, adalimumab; or a B-cell depleting, anti-CD20 antibody therapy, such as rituximab; and the combination therapies include lecanemab and adalimumab, lecanemab and rituximab, lecanemab and etanercept, or adalimumab and rituximab. AD patients are stratified based on the FcGR-3A and -2A functional polymorphisms and an AD subject is selected, for example, from Group-2. AD patients are also stratified based on plasma ATN biomarkers. In another embodiment, AD patients can be stratified for both FcGR-3A, -2A polymorphisms and ATN biomarkers. In one embodiment, adalimumab monotherapy and the combination therapy comprising lecanemab and adalimumab are administered in select subsets as stratified by the FcGR-3A, -2A polymorphisms, for example, Groups-1,2,3,4, and 7 with an A+TN signature. In another embodiment, rituximab monotherapy and the combination therapy comprising lecanemab and rituximab are administered in select subsets as stratified by the FcGR-3A, -2A polymorphisms, for instance, Groups-1,2,3,4, and 7 with an A⁺T⁺N⁻ signature. Etanercept monotherapy and the combination therapy comprising lecanemab and etanercept are administered in select subsets, for example, Groups-5,6,8, and 9 with an A⁺T⁻N⁻ signature.

Also disclosed herein are methods that include administering a monotherapy or a combination therapy to an AD patient selected from a stratified patient population in a treatment protocol, wherein: the monotherapy can be an anti-Aβ antibody, lecanemab; another option for monotherapy is an anti-TNF therapy, such as adalimumab or etanercept; another option for monotherapy is a B-cell depleting, anti-CD20 antibody therapy, such as rituximab; and the combination therapies can include lecanemab and adalimumab, lecanemab and rituximab, lecanemab and etanercept, or adalimumab and rituximab. In one embodiment, AD patients are stratified based on the FcGR-3A and -2A functional polymorphisms and an AD patient is selected, for example, from Group-2. In another embodiment, AD patients can be stratified based on ATN biomarkers. In yet another embodiment, AD patients may be further stratified based on B-cell subset populations, such as naïve, memory, and plasma subsets, as measured by minimal residual disease flow cytometry (MRD-FC). In other embodiments, AD patients can be further stratified based on the levels of inflammatory macrophages and categorized into high, medium, and low levels. In another embodiment, patients may be further stratified based on the presence of high or low levels of cytokines such as TNF-α, IL-1, IL-6, and others, in plasma. In a comprehensive approach, AD patients are stratified for FcGR-3A, -2A polymorphisms, ATN biomarkers, B-cell subsets, inflammatory macrophages, and cytokine levels. In some embodiments, adalimumab monotherapy and the combination therapy comprising lecanemab and adalimumab are administered in select subsets as stratified by the FcGR-3A, -2A polymorphisms, for example, Groups-1,2,3,4, and 7 with an A⁺T⁻N⁻ signature. Rituximab monotherapy and the combination therapy comprising lecanemab and rituximab are administered in select subsets as stratified by the FcGR-3A, -2A polymorphisms, for example, Groups-1,2,3,4, and 7. The combination therapy comprising adalimumab and rituximab is administered in select subsets as stratified by the FcGR-3A, -2A polymorphisms, for example, Groups-1,2,3,4, and 7. Etanercept monotherapy and the combination therapy comprising lecanemab and etanercept are administered in select subsets, for example, Groups-5,6,8, and 9 with an A⁺T⁻N⁻ signature.

Also disclosed herein are methods that include administering a monotherapy or a combination therapy to a subject enrolled in an AD clinical trial, wherein at least 3 clinical trial arms are run in parallel, and wherein: each of the at least 3 clinical trial arms employs a therapy, which can be a non-licensed and non-approved therapy for AD; at least one of the therapies in the 3 clinical trial arms is administered via a different delivery route, such as subcutaneous versus intravenous; and a subject remains in one of the at least 3 clinical trial arms when the therapy shows efficacy in the subject, or the subject can be moved to a different one of the at least 3 clinical trial arms when the therapy shows decreased or no efficacy. In one embodiment, the therapies used in the 3 clinical trial arms include lecanemab, adalimumab, and etanercept. In one embodiment, the AD clinical trial is aimed at patients who have any one of the underlying autoimmune or inflammatory diseases: rheumatoid arthritis, polyarticular juvenile idiopathic arthritis, Crohn's disease, ulcerative colitis, ankylosing spondylitis, psoriasis, plaque psoriasis, or psoriatic arthritis. In some embodiments, at least one clinical trial arm may include at least one additional therapy, which can be different from the first therapy. In other embodiments, therapies may be administered to the subject by a different route of administration or under a different dosing schedule. In another embodiment, each of the at least 3 clinical trial arms could be a Phase IV, Phase III, or a Phase II clinical trial, or an RWE trial. The method may also entail combining at least 3 clinical trial arms into a single clinical trial. In one embodiment, a method may include conducting a diagnostic test, which might include an in vitro diagnostic test or a theragnostic test. In another embodiment, the tests are designed to measure therapeutic and/or clinical efficacy, or to assess disease progression in AD, for instance, through longitudinal assessments of inflammatory macrophages, B-cells, subsets of B-cells, or cytokine levels on a subject-by-subject basis. Diagnostic tests might also target disease severity in autoimmune diseases, such as rheumatoid arthritis. In some embodiments, the in vitro diagnostic test may not be approved or cleared by a regulatory agency. It is noted that from about 1% to about 5% of subjects may not exhibit efficacy when a therapy is administered.

Disclosed are methods of treating an AD patient by determining a category of ARIA and CAA incidence by genotyping the individual for the FcGR-2A and/or FcGR-3A functional polymorphisms. In one embodiment, the FcGR-2A HH¹³¹ polymorphism and the FcGR-3A VV¹⁵⁸ polymorphism together indicate a high degree of ARIA/CAA incidence, while the FcGR-2A RR¹³¹ polymorphism and the FcGR-3A FF¹⁵⁸ polymorphism together indicate a low degree of ARIA/CAA incidence. In another embodiment, the plasma ATN levels are measured in that subject. It is noted that the nine subsets of patients generated by the two FcGR polymorphisms differ in ARIA and/or CAA incidence, which can be measured by either in vitro or in vivo methods, or by PET imaging. In one embodiment, such a stratification could lead to tailored dosing of the anti-Aβ antibody therapies to an AD subject to effectively reduce the incidence of ARIA. For example, lecanemab dosing for an AD subject in Group-1 could be 5-7 mg/Kg instead of a standard dosing of 10 mg/Kg. In another embodiment, such stratification and dosing protocols are employed to recruit AD patients or a subset of AD patients in a clinical trial. In another embodiment, such protocols are adopted to select an AD patient or a subset of AD patients for lecanemab or donanemab treatment.

Also disclosed are methods of determining the degree of ARIA incidence to an antibody therapy by genotyping the AD subject for one or more FcGR-3A and/or -2A functional polymorphisms and using the first and/or second FcGR polymorphisms to determine the degree of ARIA incidence in the subject due to anti-Aβ antibodies that use ADCP as a major mechanism of action, such as aducanumab, lecanemab, donanemab, or gantenerumab.

Disclosed are methods of enrolling a subject in an AD clinical trial and/or selecting a therapy in a treatment protocol for an ADCP- or ADCC-treatable disease by genotyping for the FcGR-2A functional polymorphism to stratify the patient population into three groups (HH¹³¹, HR¹³¹, RR¹³¹); genotyping for the FcGR-3A functional polymorphism to stratify the patient population into three groups (VV¹⁵⁸, VF¹⁵⁸, FF¹⁵⁸); and stratifying subjects into nine patient groups I-IX based on the first and second results. In one embodiment, this stratification is used to select a therapy, such as a biologic, or to titrate appropriate dosing or a dosing schedule for a biologic, such as lecanemab or donanemab, for the patient group ensuring the degree of ARIA incidence related to the antibody therapy in the patient group is minimized. In another embodiment, subjects are classified into nine groups based on their polymorphisms, including: VV¹⁵⁸, HH¹³¹ (Group-1); VF¹⁵⁸, HH¹³¹ (Group-2); FF¹⁵⁸, HH¹³¹ (Group-3); VV¹⁵⁸, HR¹³¹ (Group-4); VF¹⁵⁸, HR¹³¹ (Group-5); FF¹⁵⁸, HR¹³¹ (Group-6); VV¹⁵⁸ RR¹³¹ (Group-7); VF¹⁵⁸, RR¹³¹ (Group-8); and FF¹⁵⁸, RR¹³¹ (Group-9). In an embodiment, AD is treated with a biologic, such as etanercept, that does not employ ADCC as a key therapeutic mechanism. In another embodiment, AD is treated with a biologic drug such as adalimumab or rituximab, which uses ADCC as a major therapeutic mechanism. Patients are further stratified based on the plasma levels of ATN biomarkers. For example, patients in Group-1 with an A⁺T⁻N⁻ signature may be enrolled in a clinical trial. In an embodiment, patients in Group-1 may be further stratified based on B-cell subset populations, such as naïve, memory, and plasma subsets as measured by minimal residual disease flow cytometry (MRD-FC). In another embodiment, patients in Group-9 may be further stratified based on plasma levels of cytokines, such as TNF-α, IL-1, IL-6, etc.

Also disclosed herein are methods that comprise administering a monotherapy or a combination therapy to an AD subject selected from a stratified patient population and enrolled in a clinical trial, where: the monotherapy is an anti-Aβ antibody, lecanemab; or an anti-TNF therapy, either adalimumab or etanercept; or an anti-CD20 antibody therapy, such as rituximab; and where the combination therapy comprises: lecanemab and adalimumab, lecanemab and rituximab, or adalimumab and rituximab, or lecanemab and etanercept. In various embodiments, AD patients are stratified based on the FcGR-3A and -2A functional polymorphisms, plasma ATN signature, or both. In one embodiment, AD patients in Group-1 and Group-4 are further stratified based on B-cell subset populations or the levels of inflammatory macrophages, or the presence of high or low levels of cytokines such as TNF-α, and IL-6 in plasma. For instance, lecanemab monotherapy and the combination therapy comprising lecanemab and adalimumab or lecanemab and rituximab are administered to subsets stratified by the FcGR-3A, -2A functional polymorphisms with an A⁺T⁻N⁻ signature, such as Groups-1,2,3,4, and 7. In yet another embodiment, etanercept monotherapy and the combination therapy comprising lecanemab and etanercept are administered to subsets, such as Groups-5,6,8, and 9 with an A⁺T⁺N⁻ signature.

Also disclosed herein are methods that comprise administering a monotherapy or a combination therapy to an AD subject selected from a stratified patient population and enrolled in a treatment protocol, wherein: the monotherapy options can be: an anti-Aβ antibody, donanemab; an anti-TNF therapy, either adalimumab or etanercept; an anti-CD20 antibody therapy, rituximab; and the combination therapy options comprising lecanemab and adalimumab, donanemab and rituximab, adalimumab and rituximab, or donanemab and etanercept. In one embodiment, AD patients are stratified based on the FcGR-3A and -2A functional polymorphisms. Further stratification of AD patients is based on the ATN signature. In another embodiment, AD patients can be stratified for both FcGR-3A, -2A functional polymorphisms and ATN signature. In yet another embodiment, AD patients in Group-1 may be further stratified based on B-cell subset populations, such as naïve, memory, and plasma subsets, as determined by minimal residual disease flow cytometry (MRD-FC). Similarly, AD patients in Group-4 are further stratified based on the levels of inflammatory macrophages in peripheral blood and categorized into high, medium, and low populations. In one embodiment, patients in Groups 1-9 are stratified based on the presence of high or low levels of cytokines like TNF-α, IL-1, IL-6, in plasma. In an yet another embodiment, adalimumab monotherapy or the donanemab and adalimumab combination therapy is administered in designated subsets as stratified by the FcGR-3A, -2A polymorphisms, for example, Groups-1,2,3,4, and 7 with an A⁺T⁺N⁻ signature. In some embodiments, etanercept monotherapy and the etanercept and donanemab combination therapy are administered in other subsets, for example, Groups-5,6,8, and 9 with an A⁺T⁻N⁻ signature.

Also disclosed is a method of conducting a classical clinical trial in an unstratified, all-comers population of AD subjects, comprising: enrolling subjects diagnosed with AD without prior stratification based on mechanism of action of therapies, mechanism of disease severity; randomly assigning the enrolled subjects to receive at least one therapy selected from the group consisting of: an anti-Aβ antibody as a monotherapy, an anti-TNF biologic as a monotherapy, a B-cell depleting antibody as a monotherapy, a combination therapy of an anti-Aβ antibody and an anti-TNF biologic, a combination therapy of an anti-Aβ antibody and a B-cell depleting antibody, or a combination therapy of an anti-TNF biologic and a B-cell depleting antibody; monitoring the subjects for changes in cognitive and functional outcomes over a predetermined trial period; and comparing the efficacy and safety of the administered therapies across the unstratified AD population to identify effective treatment(s) for AD. In one embodiment, these are conventional, all-comers trials where patients are not stratified based on the mechanism of action of drugs or disease severity in trial enrollment decisions or determination of degree of responsiveness. In another embodiment, such trials may utilize PET imaging data or measure plasma or cerebrospinal fluid (CSF) levels of ATN biomarkers. In yet another embodiment, the method further comprises analyzing the trial data retrospectively to identify one or more patient subsets based on genetic, inflammatory, and/or immune biomarker profiles that demonstrate better or worse treatment responsiveness to the therapies. In one embodiment, the method further comprises revising the product label for the at least one therapy, based on the identified patient subsets, to include specific indications, contraindications, dosage adjustments, or warnings directed to the patient subsets demonstrating better or worse treatment responsiveness.

Also disclosed is a method of administering a classical treatment protocol in an unstratified, all-comers population of AD patients, comprising: identifying patients diagnosed with AD without prior stratification based on mechanism of action of therapies, mechanism of disease severity; administering to the patients a therapy selected from the group consisting of: an anti-Aβ antibody as a monotherapy, an anti-TNF biologic as a monotherapy, a B-cell depleting antibody as a monotherapy, a combination therapy of an anti-Aβ antibody and an anti-TNF biologic, a combination therapy of an anti-Aβ antibody and a B-cell depleting antibody, or a combination therapy of an anti-TNF biologic and a B-cell depleting antibody; monitoring therapeutic outcomes and side effects in the treated patients over a predetermined treatment period; and adjusting the therapy based on the observed efficacy and side effects in the patients, aiming to optimize therapeutic outcomes in the unstratified AD population. In one embodiment, these are conventional, all-comers treatment protocols where patients are not stratified based on the mechanism of action of drugs or disease severity for treatment decisions including determination of degree of treatment responsiveness. In one embodiment, such protocols may utilize PET imaging data or measure plasma or cerebrospinal fluid (CSF) levels of ATN biomarkers.

The terms “administration,” “co-administration,” “administered in combination with” and their grammatical equivalents or the like, as used herein, can encompass administration of therapies to a subject, including treatment regimens in which therapies, such as a combination therapy, are administered by the same or different route of administration or at the same or different times, for instance, lecanemab being administered intravenously and adalimumab being administered subcutaneously. In one scenario, a combination therapy comprising two therapies may use the same mechanism as the primary mechanism of action or use at least two distinct mechanisms of action. Additionally, a therapy may be co-administered with other agents, for example, small molecule therapies such as methotrexate. These terms encompass administration of one or more therapies to a subject so that therapies and/or their metabolites are present in the subject simultaneously, at staggered times, or within a composition where one or more therapies are present. Thus, in one embodiment, medicament(s) may be administered in a single composition. Alternatively, medicament(s) can be admixed in the composition. In some scenarios, the same therapy may be administered via a combination of different routes of administration. Furthermore, medicament(s) administered can be in a therapeutically effective amount.

Also disclosed herein are methods of increasing either safety or efficacy, or both safety and efficacy of a therapy, such as lecanemab, adalimumab, etanercept, or rituximab, which include employing a method of administering as detailed herein.

Disclosed herein is computer-readable memory that stores, at least transiently, on an electronic storage device, a database comprising clinical trial data, or a summary thereof, in computer-readable format obtained from a clinical trial in AD using a method of administering as described herein. In some embodiments, a database further comprises diagnostic clinical trial data in AD. Additionally, a diagnostic clinical trial data may include theragnostic data. Moreover, a database may further comprise AD clinical trial data of a device, including brain imaging data. In some embodiments, a system might not require regulatory clearance, licensing, or approval by a regulatory agency. Possible regulatory agencies include the US Federal Drug Administration (FDA), the European Medicines Agency (EMA), the Medicines and Healthcare Products Regulatory Agency (MHRA) in the U.K., the Chinese FDA, and the Pharmaceuticals and Medical Devices Agency (PMDA) in Japan. In some embodiments, a system has previously been exempted, cleared, licensed, or approved by a regulatory agency.

Also disclosed herein is a computer-readable memory storing, at least transiently, on an electronic storage device an electronic regulatory submission (ERS), or a section thereof, in computer-readable format. In one embodiment, the electronic regulatory submission contains clinical trial data or a summary thereof, for example, obtained from a clinical study or trial, such as RWE trial in AD, incorporating a method of administering as described herein. In another embodiment, the submission may specify the steps of a method of administering as described herein.

Also disclosed herein is computer-readable memory storing, at least transiently, on an electronic storage device, a regulatory submission application in computer-readable format seeking approval for a drug label. In one embodiment, the regulatory submission application contains clinical trial data, or a summary thereof, obtained from a clinical trial in AD employing a method of administering as described herein. In another embodiment, the AD clinical trial data comprises data from a diagnostic, and the label revision requires administration of the theragnostic evaluation before the medicament is administered on a subject-by-subject basis.

Also disclosed herein is a computer-readable memory storing on an electronic storage device a regulatory submission application in computer-readable format seeking approval for label revision of a drug. The regulatory submission application details the steps of a method of administering therapies in stratified subset(s) of patients, or by patient-by-patient basis as described herein. In one embodiment, the revision includes a label expansion or label restriction. In another embodiment, the label includes a new drug label being designated for a new indication, for example, administration of rituximab, adalimumab, or etanercept for treating AD. In one embodiment, a drug label might specify a new mechanism of action of a drug to treat a new indication, such as administration of rituximab to target B-cells in the brain or peripheral blood to treat AD; the use of adalimumab or etanercept to target either proinflammatory immune cells such as macrophages, neutrophils, or NK-cells, or the cytokines such as TNF-α or IL-6 in peripheral blood to treat AD; or administration of lecanemab to facilitate clearance of Aβ plaques in peripheral blood through macrophage- or neutrophil-mediated ADCP. In another embodiment, the label revision may require a theragnostic evaluation step to stratify patients for treatment decisions, which could include measurement of B-cell subset populations, such as naïve, memory, and plasma subsets, by minimal residual disease flow cytometry (MRD-FC), or assessing the levels of inflammatory macrophages in peripheral blood and the levels of cytokines such as TNF-α, IL-1, IL-6, in plasma.

Also disclosed herein are kits that comprise a drug and an approved drug label, such as a label revision, approved based in part on a regulatory agency evaluation utilizing computer-readable memory as described herein. In an embodiment, a container comprises a drug.

Also disclosed herein are kits that comprise a drug in a container and a package insert. In an embodiment, the package insert contains the data, a summary thereof, or the steps thereof, of a method of administering as described herein, or any combination thereof.

Also disclosed herein is a label-revised drug that was developed by conducting a method of administering as described herein. In one embodiment, the label revision is a new label to treat a new indication. In another embodiment, the label revision for a drug is approved by a regulatory agency for treating CU individuals with SCD (CU-SCD) and/or MCI patients, where the drug could be an anti-TNF therapy, such as etanercept or adalimumab. In yet another embodiment, the new label for a drug may be generated by a third-party trial sponsor and approved by a regulatory agency for treating AD, where the drug is a B-cell depleting anti-CD20 therapy, such as rituximab. In some embodiments, prior authorization from a payer is required to dispense and administer the drug in AD. For example, the label-revised drug being lecanemab monotherapy for treating AD in stratified subset(s) of patients, such as Groups-1,2,3,4, and 7 with an A+T-N-signature, aims to achieve significantly better clinical outcomes, for example, an improvement in CDR-SB score of 1.5-2.0 versus an improvement of 0.5 in an all-comers treatment setting. In one embodiment, the new label generated for etanercept targets treating MCI patients or CU-SCD individuals. In another embodiment, the new label for etanercept in AD becomes a class label covering etanercept and all approved etanercept biosimilars.

Also disclosed herein is a computer-readable memory storing on an electronic storage device a drug formulary, in computer-readable format, which comprises a label-revised drug as described herein. In an embodiment, prior authorization from a payer is required to dispense the label-revised drug from a specialty pharmacy.

Also disclosed herein is a computer-readable memory storing on an electronic storage device a drug formulary, in computer-readable format, which comprises a medicament not necessarily licensed or approved by a regulatory agency for treating AD, but which is licensed and approved by the same or a different regulatory agency for treating another indication, such as adalimumab for rheumatoid arthritis. In some embodiments, a medicament is only authorized for administration in a clinical trial. In one embodiment, the medicament could be an etanercept biosimilar.

Also disclosed herein are systems that comprise a computer-readable memory as described herein and a computer processor. In one embodiment, this computer processor is configured to access the drug formulary from the computer-readable memory. In another embodiment, the system further comprises a communication device operatively coupled to it.

Also disclosed herein are pharmacies, such as specialty pharmacies, which comprise a system as described herein and a physical storage of medicaments, with at least some of the medicaments in the physical storage being recited in the system. In some embodiments, the physical storage of medicaments and the system are both present in the same building. In an embodiment, a system may be configured for access within the same building as the physical storage. In another embodiment, the medicaments may include aducanumab, lecanemab, donanemab, rituximab, adalimumab, etanercept, an adalimumab biosimilar, and an etanercept biosimilar.

Also disclosed herein are pharmacies that comprise a drug that has received a new label or a label-revision, as described herein, in a container. In an embodiment, a pharmacy, such as a specialty pharmacy, may carry a medicament that is not licensed or approved by a regulatory agency for treating AD. In one embodiment, a medicament could be a biosimilar, such as an etanercept biosimilar, that is approved and licensed for treating AD. In an embodiment, a pharmacy could comprise a medicament categorized as a specialty drug, which is intended to treat complex, chronic, rare, or particularly difficult to manage diseases or disorders, like AD, rheumatoid arthritis, multiple sclerosis, etc. In another embodiment, pharmacies may comprise medicaments that are interchangeable with medicaments that have been approved and licensed. An example of such a medicament is an anti-TNF therapy, like an interchangeable adalimumab biosimilar.

Also disclosed herein are methods that comprise consulting a system as described herein to access the drug formulary; performing an assay on a sample from a subject, or using data derived therefrom, to stratify responders to a therapy based on a major mechanism of action of a drug, and further stratifying AD patients by disease stage and/or disease type; and providing prior authorization. In one embodiment, the prior authorization comprises enrolling the subject in an AD clinical trial to receive a non-licensed and non-approved medicament recited in the formulary from a pharmacy; or providing a treatment to subjects not participating in the clinical trial; or providing a treatment to subjects who are participating in the RWE clinical trial. In another embodiment, this prior authorization is provided based at least in part on the assay or data derived therefrom and is provided on a patient-by-patient basis.

Also disclosed herein are methods that comprise consulting a system as described herein and selecting a medicament to be administered to a subject in a treatment or clinical trial protocol. In some embodiments, a clinical trial sponsor or provider, who may be located at a different location than the subject, consults the system. In an embodiment, a method may further comprise a process of administering the medicament to the subject. In some embodiments, a specialty drug distributor may not be included in the process of administering the medicament to the subject. In another embodiment, a pharmacy benefit manager (PBM) may not be included in the process of administering the medicament to the subject.

Also disclosed herein are methods of treating, slowing, halting, or reversing the progression of AD that comprise employing a method of administering as described herein.

Also disclosed herein are methods of treating, slowing, halting, or reversing the progression of AD that comprise employing a system as described herein.

Also disclosed herein are methods of treating, slowing, halting, or reversing the progression of AD that comprise employing a computer-readable memory as described herein.

Also disclosed herein are methods that comprise administering a non-approved and non-licensed medicament in AD by employing a method of administering as described herein. In one embodiment, this includes a medicament that was investigated in a clinical trial, such as in Phase-3 but discontinued; and comprises generating data to obtain a drug label or licensing for the non-licensed and non-approved medicament based at least in part on a clinical trial. In certain embodiments, the medicament was approved and licensed by a regulatory agency, but further commercialization of the drug, such as an Aβ-targeting antibody, was discontinued.

Also disclosed herein are methods that can comprise administering a medicament to a subject enrolled in an AD clinical trial, which may be conducted in the US; and where the medicament is a specialty drug approved for the treatment of AD. In one embodiment, the specialty drug is lecanemab. In another embodiment, the specialty drug is adalimumab. In yet another embodiment, the specialty drug is a combination therapy comprising lecanemab and adalimumab, lecanemab and etanercept, or donanemab and rituximab. In yet another embodiment, a provider of the clinical trial is paid at least about $500 in medicament cost for a month of treatment on a subject-by-subject basis only if the medicament demonstrates efficacy, with the drug cost being covered by a third-party payer. In other embodiments, a clinical trial can be a prospective or an RWE trial. In some embodiments, a clinical trial is conducted by a third-party sponsor. In some embodiments, clinical trial costs are at least partially covered by a third-party payer, such as Medicare or Veterans Health Administration in the US, or NHS-England in the UK. In some embodiments, the AD clinical trial enrolls at least about 10,000, 100,000, or even 1,000,000 subjects.

Also disclosed herein are methods of gaining exemption from infringement of a US patent with claims covering a patented technology. In one embodiment, these methods comprise using the patented technology solely for uses reasonably related to the development and/or submission of information, such as non-routine submission of data or results, under Federal law that regulates the manufacture, use, offer to sell, or sale of drugs or veterinary biological products (Ching E P and Ramakrishnan V, PCT/US2019/018811). In some embodiments, such use reasonably related to the development or submission of information under Federal law pertains to a clinical trial. In another embodiment, a method can comprise administering a medicament to a subject in a clinical trial within the US, with the clinical trial participation cost being paid by a third-party payer on a subject-by-subject basis depending on efficacy. In another embodiment, payment scenarios could include a pay-as-you-go basis or in multiple installments, even if at least one subject does not show efficacy. The medicament could be an anti-Aβ antibody, an anti-TNF biologic, an anti-CD20 antibody, or a biosimilar thereof. In another embodiment, the trial sponsor can be a party who does not at least partially own or have licensed intellectual property to the medicament, its formulation, or its method of use. In certain embodiments, the financial and/or legal liabilities related to the clinical trial are assumed by the third-party sponsor. In another embodiment, the trial sponsor and the payer are the same entity.

Also disclosed herein are methods of gaining exemption from infringement of a European or other patent with claims covering a patented technology. In one embodiment, these methods can comprise using the patented technology in a clinical trial solely for uses reasonably related to the development or submission of information under a Bolar Exemption, which regulates the manufacture, use, offer to sell, or sale of drugs. In some embodiments, a clinical trial may be conducted in Europe. In some embodiments, the patented drug includes Aβ-targeting antibodies, such as aducanumab, lecanemab, donanemab, or gantenerumab.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### Alzheimer's Disease

Alzheimer's disease (AD) is a complex, progressively debilitating, and fatal neurodegenerative disease. AD is rapidly increasing in frequency as the world's population ages. There are currently an estimated 6.5 million individuals with AD in the US, and this number is expected to increase to more than 13 million by 2050. Approximately 15% of the US population over age 60 is diagnosed with prodromal AD and approximately 40% are in the preclinical stages. Similar trends are seen globally with an anticipated worldwide population of AD dementia patients exceeding 100 million by 2050 if effective means of delaying, preventing, or reversing AD are not developed. There is a significant need for developing therapeutics that could halt or reverse the underlying pathology of AD.

Across all types of AD therapies, the failure rate in clinical development exceeds 99%, and for disease-modifying therapies (DMTs), the failure rate reaches 100%. This underscores the need for a re-examination of the drug development process.

AD patients exhibit heterogeneity; they can be in the preclinical AD continuum spanning up to two decades or more without exhibiting any clinical symptoms. Cognitively unimpaired (CU) individuals and CU individuals with subjective cognitive decline (SCD) are in the preclinical phase of the AD continuum, whereas patients who exhibit mild cognitive impairment (MCI), functional decline, and AD dementia are classified under the clinical phase of the AD continuum. Furthermore, misdiagnosis of AD patients is common, with 10-30% of individuals clinically diagnosed with AD dementia found not to have AD neurodegeneration at autopsy.

The beta-amyloid (Aβ) cascade hypothesis is one area actively pursued to develop passive immunotherapies. The accumulation of Aβ peptide in the form of oligomers, protofibrils, or insoluble Aβ plaques in the brain is an early event in disease pathogenesis. The AB plaque formation triggers the onset and further progression of AD through the deposition of tau tangles and neurodegeneration. According to a revised biological definition of AD, regardless of the presence of tau tangles (T⁻) or neurodegeneration (N⁻), the presence of Aβ (A⁺) alone places a patient in the AD continuum (Jack C R Jr, et al. Alzheimers Dement. 2018;4:535). Based on this hypothesis, the therapeutic intervention strategy seeks faster and complete clearance of Aβ plaques and soluble aggregates, which could presumably improve cognition and/or daily function, or potentially halt the progression of AD.

Cognitively unimpaired (CU) individuals with a low burden of Aβ pathology, even before the presence of overt Aβ deposition in the brain, exhibit typical AD pathophysiological changes. Specifically, the main AD CSF biomarkers, reflecting tau, synaptic, and neurodegenerative changes, are already altered at this stage, and structural and functional brain changes are either minimal or absent. Plasma biomarkers such as p-tau231 and p-tau217 better capture the earliest cerebral Aβ changes, prior to overt Aβ plaque pathology sets in, and are thus considered promising blood biomarkers to enrich a preclinical population for AD clinical trials (Milà-Alomà M, et al. Nat Med. 2022;28:1797-1801) and treatment. Furthermore, a subset of CU individuals might present subjective cognitive decline (SCD; Cullen N C, et al. Nat Commun. 2021;12:3555) with an A⁺T⁻N⁻ signature.

By leveraging on the amyloid cascade hypothesis, several passive immunotherapies aim to treat AD by targeting the Aβ peptide and plaques, or tau protein. These drug candidates have generally failed to achieve clinically effective outcomes or have at best provided minimal improvements in clinical trials. The FDA has used the Aβ plaque clearance in the brain as the surrogate endpoint for aducanumab's accelerated approval for treating early AD and MCI patients. Yet, in two identical clinical trials, aducanumab yielded contradicting results; its clinical efficacy in preventing the disease progression remains inconclusive (Budd Haeberlein S, et al. J. Prev. Alzheimers Dis. 2022;9:197). The EMA has opted not to approve aducanumab in Europe. The effectiveness of clearing either Aβ, tau, or both as a therapeutic intervention to significantly impact clinical outcomes is still uncertain, and whether this approach is effective in all AD patients or only in some subset(s) is yet to be determined.

Lecanemab is the second drug to be approved in this class by FDA, and donanemab is likely to be approved soon.

### Autoimmune and Inflammatory Diseases and AD

Systemic autoimmune and inflammatory diseases are partly caused in part by the production of TNFα by activated (inflammatory) macrophages in peripheral blood or disease specific compartments such as the synovium; anti-TNF agents are effective in treating several autoimmune and inflammatory diseases such as rheumatoid arthritis, polyarticular juvenile idiopathic arthritis (jIA), Crohn's disease, ulcerative colitis, ankylosing spondylitis, psoriasis, plaque psoriasis, and psoriatic arthritis. Inflammation in RA is notably pronounced in the synovial lining of the joints where synovial cell proliferation leads to the thickening of the lining, infiltration by activated macrophages, B-cells, and other inflammatory cells, elevated production of TNFα, and irreversible destruction of the joint architecture and function (Keffer J, et al. EMBO J. 1991; 10:4025-31).

Systemically produced TNF-α in peripheral blood may enter the brain through receptor-mediated transcytosis (Pan W and Kastin A J, Exp. Neurol. 2002; 174:193-200). In mice, both types of TNF receptors, TNFR1 and TNFR2, participate in the transport of TNF-α across the blood-brain barrier (BBB). Thus, even with an intact BBB, TNF-α produced systemically by inflammatory macrophages, neutrophils, NK-cells, and B cells may infiltrate the brain and contribute to AD pathogenesis.

Systemic inflammatory disease is a substantial risk factor for AD. Inflammatory bowel disease (IBD), Crohn's disease, and RA were associated with over a two-fold higher risk for AD (adjusted odds ratio [AOR] 2.06-2.46). The increased risk associated with systemic inflammation was also replicated in AD dementia patients, with the risk for AD dementia elevated two-fold in patients with RA, IBD, ankylosing spondylitis, ulcerative colitis, and Crohn's disease (AOR 2.00-2.69). Psoriasis was also associated with a higher risk for AD and dementia although to a lesser extent (AORs of 1.37 and 1.49, respectively). Systemic inflammation is linked with a higher risk for AD and AD dementia across multiple inflammatory diseases involving the joints, the gut, and the skin (Zhou M, et al. PLOS One. 2020; 15:e0229819).

Though AD is generally classified as a neurological disorder affecting the brain, given that both adaptive and innate immune responses play significant roles in AD pathology, it can also be characterized as an autoimmune disease or an inflammatory disorder.

### Blood Brain Barrier

The blood-brain barrier (BBB) is formed by a tightly sealed monolayer of brain endothelial cells, which prevents neurotoxic plasma-derived components, RBCs, leukocytes, and pathogens from entering the central nervous system (CNS). An early breakdown and/or dysfunction of the BBB have been shown in AD well before the onset of dementia, neurodegeneration, and/or brain atrophy (Montagne A, et al. J. Exp. Med. 2017; 214:3151-69). Such breaching enables to the bidirectional flow of immune cells, cytokines, and other proteins (for example, Aβ plaques, tau protein, NfL), etc. between the brain and peripheral blood. Therefore, in one embodiment, this breached state can be exploited to target novel immune mechanisms both in the brain and peripheral blood to develop effective therapies.

The occurrence of infiltrated macrophages (Hultman K, et al. J. Cereb. Blood Flow Metab. 2013; 33:1251-58) and neutrophils in the brain in human AD indicates that the BBB has been compromised, leading to the influx of leukocytes into the brain. Neutrophils were found adhered to and spread inside brain venules and were also located in the parenchyma, accompanied by neutrophil extracellular traps (NETs). In transgenic AD models, neutrophil depletion or inhibition of neutrophil trafficking was shown to reduce AD-like neuropathology and to improve memory in mice already exhibiting cognitive dysfunction. The temporary depletion of neutrophils for 1 month at early stages of disease resulted in sustained improvements in memory. Transgenic AD model mice lacking lymphocyte function-associated antigen 1 (LFA-1) showed protection from cognitive decline and exhibited reduced gliosis. These findings suggest that infiltrated neutrophils play a significant role in AD pathogenesis and cognitive impairment (Zenaro E, et al. Nat. Med. 2015;21:880-6).

Natural killer (NK) cells secrete inflammatory cytokines such as interferon-gamma and TNF-α, as evidenced by the increased proinflammatory profile of NK cells in triple-transgenic AD (3xTg-AD) mouse model. Depletion of NK cells led to improved cognitive function of 3xTg-AD mice. Microglia in NK-cell depleted 3xTg-AD mice exhibited a homeostatic-like morphology and a decreased expression of multiple proinflammatory cytokines. This suggests that NK cells contribute to neuroinflammation and AD-associated cognitive decline (Zhang Y, et al. J. Immunol. 2020;205:502).

Microglia, the resident macrophages in the brain, are activated by neuroinflammatory changes around the Aβ plaques, resulting in increased levels of proinflammatory mediators such as TNF-α, which in turn can reactivate microglia and potentially contribute to AD pathogenesis. Additionally, immune cells such as inflammatory macrophages, neutrophils, and B-cells in peripheral blood are key drivers of systemic inflammation through overproduction of TNF-α and other proinflammatory cytokines such as IL-1 and IL-6. Therefore, targeting any one of these cytokines could represent an effective therapeutic drug development strategy.

ATN biomarkers are known to migrate from the brain to peripheral blood, and this translocation is believed to be more pronounced in a BBB-breached state. 40-60% of Aβ generated in the brain could diffuse into the blood and be cleared peripherally, highlighting that the peripheral immune cells' crucial role in Aβ plaque management (Chen S-H, et al. Transl. Psychiatry 2020;10:423). Similar to microglia-mediated phagocytosis of Aβ plaques in the brain, this invention discloses an independent therapeutic mechanism of action in peripheral blood, wherein macrophages and/or neutrophils can clear Aβ plaques and oligomers in the presence of passive immunotherapies such as aducanumab, lecanemab, or donanemab. In one embodiment, targeting the phagocytosis of plaques in peripheral blood by macrophages or neutrophils with anti-Aβ antibody therapies is an effective strategy to slow, halt, or reverse disease progression, or to achieve clinical remission. An additional therapeutic strategy to treat AD is to target the pathophysiology of microglial inflammation in the brain, particularly in a BBB-breached state. In one embodiment, anti-TNF therapies delivered through subcutaneous or intravenous routes can target immune cells, such as inflammatory macrophages, neutrophils, and natural killer (NK) cells, as well as inflammatory mediators and cytokines secreted both in the brain and blood. For example, an anti-TNF therapy like etanercept, adalimumab, or infliximab may offer an effective therapeutic strategy to slow, halt, or reverse disease progression, or to achieve clinical remission. However, it is not known whether this monotherapy approach alone could serve as an effective treatment strategy for AD. Another therapeutic strategy to treat AD focuses on targeting the adaptive immune response, such as by targeting B-cells both in peripheral blood and the brain. A B-cell depleting, anti-CD20 antibody therapy such as rituximab delivered through subcutaneous or intravenous routes, could potentially be effective in slowing, halting, or reversing the progression of AD. However, it is not known whether this monotherapy approach could serve as an effective treatment strategy for AD.

In an embodiment, an effective therapeutic intervention could be monotherapy or combination therapy targeting one or more mechanisms in AD. In one embodiment, such a combination therapy may include administration of donanemab and an anti-TNF biologic, like adalimumab. In another embodiment, such a combination therapy in AD might include lecanemab and a B-cell depleting therapy, such as rituximab. In one embodiment, a combination therapy in AD could entail administering donanemab and an anti-TNF therapy, like etanercept. In yet another embodiment, a combination therapy could include a combination of an anti-TNF therapy and a B-cell depleting therapy. In another embodiment, an effective treatment strategy aims to target proinflammatory macrophages, NK-cells, neutrophils, and/or B-cells, wherein the therapies may comprise anti-TNF therapies (for example, infliximab, golimumab, certolizumab pegol); anti-IL-1 therapies (for example, anakinra, canakinumab); and anti-IL-6 therapies (for example, tocilizumab, sarilumab, sirukumab), among others.

### Plasma Biomarkers in AD

Aβ is thought to potentiate the spread of neocortical tau pathology which in turn drives neurodegeneration and cognitive decline (Jack C R, Jr., Holtzman D M, Neuron 2013;80:1347). These changes are slow and protracted, however, and precede the initial symptoms of AD by several years or decades (Insel P S, et al. Neurology. 2019;93:e322-33). In one embodiment, identifying individuals in the preclinical phase of the AD continuum at risk of progressing to AD dementia is crucial for developing therapeutic interventions.

The National Institute on Aging and Alzheimer's Association has proposed a framework for research wherein cerebrospinal fluid (CSF) and imaging (magnetic resonance and positron emission tomography (PET)) based measures of Aβ (A), tau (T), and neurodegeneration (N) are compiled into an ATN classification system (Jack C R, Jr., et al. Alzheimers Dement. 2018;14:535). CSF measures include the Aβ42/Aβ40 ratio, tau phosphorylated at threonine 181 (P-tau181) and neurofilament light (NfL). Sensitive methods for measuring ATN biomarkers in blood have been developed (Teunissen C E, et al. Lancet Neurol. 2022; 21:66; Hansson O, et al. Alzheimers Dement. 2022;18:2669).

Blood-based AD biomarkers, with their lower cost and ease of accessibility, may overcome the limitations inherent to CSF analysis, and PET or MRI imaging. While recent work has focused on individualized risk assessment in patients with MCI (van Maurik I S, et al. Lancet Neurol. 2019;18:1034), it is shown that substantial irreversible neuronal loss occurs at this stage, potentially diminishing the likelihood of disease-modifying therapies to prevent AD dementia onset (Sperling R A, et al. Sci. Transl. Med. 2011;3:111). This insight has spurred research into CU older individuals at risk for progression to AD dementia based on biomarker evidence of brain AD pathology (Sperling R A, et al. Sci. Transl. Med. 2014;6:228).

Studies combining blood-based Aβ42/Aβ40, P-tau, and NfL have confirmed their diagnostic accuracy for the separation of AD dementia from CU individuals and patients with non-AD disorders (Palmqvist S, et al. JAMA Neurol. 2019; 76:1060; Thijssen E H, et al. Nat. Med. 2020; 26:387).

The usefulness of plasma Aβ42/Aβ40, P-tau217, and NfL, either separately or in combination, for predicting longitudinal cognitive decline and clinical outcomes in a CU population comprising both cognitively normal (CN) healthy controls and individuals with subjective cognitive decline (SCD) who fall short of MCI diagnostic criteria has been evaluated. A combination of ATN plasma biomarkers could enhance the prediction of cognitive decline and risk of AD dementia beyond basic demographic information. Additionally, using plasma biomarkers could reduce the sample size required to conduct AD clinical trials in CU elderly populations (Cullen N C, et al. Nat Commun. 2021;12:3555).

### Passive Immunotherapies in AD

Aducanumab is a fully human IgG₁ monoclonal antibody that selectively targets aggregated Aβ. In patients with prodromal or mild AD, one year of monthly intravenous infusions of aducanumab reduced brain Aβ in a dose-and time-dependent manner. Aducanumab does not bind to soluble Aβ monomers. Aducanumab significantly increased recruitment of Iba-1⁺ microglia to Aβ plaques, demonstrating that FcGR-mediated phagocytosis (ADCP) of antibody-Aβ complexes is the clearance mechanism (Sevigny J, et al. Nature 2016;537:50).

Donanemab is a humanized IgG₁ monoclonal antibody directed at an N-terminal pyroglutamate Aβ epitope that is present only in established plaques. In a Phase-2 clinical trial, a subset of symptomatic early AD patients had complete plaque clearance at 24 weeks, improved cognition, and daily functions. These patients were able to stop or reduce the drug dose and further experienced a significant decrease in the spread of tau tangles in the brain at 76 weeks (Mintun M A, et al. New Engl. J. Med. 2021;384:1691). Donanemab utilizes ADCP as the plaque clearance mechanism.

Gantenerumab is a fully human IgG₁ monoclonal antibody that binds specifically to Aβ plaques. Gantenerumab treatment led to a dose-dependent reduction of Aβ in the brain through a microglia-dependent ADCP mechanism (Ostrowitzki S, et al. Arch Neurol. 2012;69:198). However, it failed in two separate pivotal clinical trials.

Lecanemab is a humanized IgG₁ monoclonal antibody that binds to soluble Aβ aggregates (oligomers and protofibrils) with high selectivity over the monomers and insoluble fibrils. A Phase-2b randomized clinical trial demonstrated reduction in brain amyloid accompanied by a consistent reduction of clinical decline across several clinical and biomarker endpoints (Swanson C J, et al. Alzheimer's Res. Ther. 2021;13:80). In an all-comers Phase-3 clinical trial, lecanemab reduced Aβ protofibrils in early AD and led to moderately less decline on measures of cognition and function than placebo (van Dyck C H, et al. N Engl J Med 2023;388:9). The plaque clearance mechanism mediated by lecanemab is ADCP.

### Anti-TNF Biologic Therapies

Etanercept is an anti-TNF biologic therapy commonly used to treat rheumatoid arthritis, ankylosing spondylitis, juvenile idiopathic arthritis, plaque psoriasis, and psoriatic arthritis. It is an Fc fusion protein, where the TNF receptor is fused to the Fc region of the IgG₁ antibody. It binds both TNF-α and TNF-β to inhibit the inflammatory response in joints and skin that is characteristic of these autoimmune disorders. The drug can be used as a monotherapy or be taken with other immunosuppressants, such as methotrexate (Davis et al. Arth. Rheum. 2003; 48:3230-6). Examples of biosimilars approved for use in the US or EU include: Erelzi (etanercept-szzs), and Eticovo (etanercept-ykro). It is administered subcutaneously.

Adalimumab is an anti-TNF human IgG₁ monoclonal antibody therapy approved to treat rheumatoid arthritis, juvenile idiopathic arthritis, psoriatic arthritis, ankylosing spondylitis, adult Crohn's disease, pediatric Crohn's disease, ulcerative colitis, plaque psoriasis, hidradenitis suppurativa, and uveitis. It is administered as a subcutaneous injection. Examples of biosimilars approved for use in the US or EU include: Amgevita, Solymbic, Imraldi, Cyltezo, Halimatoz, Hefiya, Hyromoz, Hulio, Idacio, Kromeya, Hadlima, Abrilada, and Amsparity.

Infliximab is yet another anti-TNF chimeric IgG₁ monoclonal antibody therapy approved for the treatment of Crohn's disease, ulcerative colitis, rheumatoid arthritis, ankylosing spondylitis, psoriasis, psoriatic arthritis, and Behçet's disease. It is administered intravenously. Examples of biosimilars approved for use in the US or EU include: Inflectra, Remsima, Renflexis, Flixabi, Ixifi, Zessly, and Avsola.

Anti-TNF therapies vary in their primary choice of mechanism of action that they use (Horiuchi T, et al. Rheumatology 2010;49:1215). Etanercept's primary mechanism is neutralization of soluble TNF-α whereas infliximab and adalimumab use ADCC as a key therapeutic mechanism by binding to transmembrane TNF-α. The Fc region of these therapies bind to FcGR-3A and -2A receptors and induce potent ADCC (Arora T, et al. Cytokine 2009;45:124). Thus, in one embodiment, in the FcGR stratified subsets, better therapeutic outcomes for adalimumab and etanercept could be expected in Groups-1-4, 7 and Groups-5,6,8, and 9, respectively.

In rheumatoid arthritis monotherapy trials, etanercept and adalimumab provided excellent response or remission in 16-22% of all-comers cohorts (Genovese M C et al. Arth. Rheum. 2002;46:1443; Gabay C, et al. Lancet 2013;381:1541). In AD trials involving RA patients, disease-specific scores, for instance, American College of Rheumatology (ACR) score in RA and CDR-SB can be used to measure clinical responses in RA and AD, respectively. Given their distinct therapeutic mechanisms, excellent responders to adalimumab and etanercept are anticipated to be in non-overlapping subsets when stratified by the FcGR functional polymorphisms. Currently, anti-TNF therapies are not approved to treat AD. In a small Phase 2 clinical trial, in mild-moderate AD dementia patients, etanercept failed to improve cognition, behavior, or global function (Butchart J, et al. Neurol 2015;84:2161-68). This was a significantly smaller trial (N=41) conducted in an unstratified, all-comers population.

### Macrophages and Microglia

Microglia, the resident macrophages in the brain, derive embryonically from the same myeloid stem cells in bone marrow that give rise to monocytes and macrophages elsewhere in the body (Ginhoux F, et al. Science. 2010;330:841). Macrophages are one of the important drivers of systemic inflammation through the overproduction of TNFα and other proinflammatory cytokines (Willrich M A, et al. Transl. Res. 2015;165:270). Multiple lines of evidence indicate that TNFα may trigger or amplify aberrant microglia signaling in the brain (Liddelow S A, et al. Nature 2017;541:481) and thereby contribute to AD pathogenesis.

Microglia are the largest population of immune cells in the central nervous system (CNS), making up 5-15% of all cells in the adult brain (Ginhoux F, et al. Science 2010;330:841). Under physiological conditions, these cells are located within the brain parenchyma, where they are in direct contact with neural progenitors, neurons, and other glial cells, namely, astrocytes and oligodendrocytes (Thion M S, et al. Science 2018;362:185). When an injury or infection takes place, microglia are recruited to the site where they engulf invading pathogens and extracellular debris by phagocytosis, supporting the normal function and integrity of the brain. In response to an inflammatory stimulus, microglia secrete a number of molecules such as proteinases, nitric oxide, reactive oxygen intermediates and pro-inflammatory cytokines, including interleukin-1 beta (IL-1β), interleukin-6 (IL-6), and TNF-α (Perry V H and C. Holmes, Nat Rev Neurol 2014;10:217).

TNF-α is a major Th1-class pro-inflammatory cytokine, produced by a variety of immune cells, including macrophages, neutrophils, microglia, and B-cells. In the CNS, activated microglia are one of the main sources of TNF-α. At basal levels, TNF-α has an important role in brain development, particularly by influencing hippocampal development and function. However, under certain pathological conditions, increased levels of TNF-α overactivate microglia, which then causes neuronal damage such as demyelination and/or neuronal degeneration. Overactivated microglia release cytotoxic molecules, including TNF-α, which are produced by a positive feedback mechanism of autocrine activation (Kuno R, et al. J. Neuroimmunol 2005;162:89). Thus, in one embodiment, by targeting overactivated microglia, anti-TNF therapies can be developed to treat AD, Parkinson's Disease (PD), multiple sclerosis (MS), amyotrophic lateral sclerosis (ALS), Huntington's Disease (HD), and frontotemporal dementia (FTD).

Microglia are also implicated in spreading tau aggregates through endocytosis and phagocytosis as well as exocytosis through exosomes in a non-synaptic transmission pathway. Tau propagation can be suppressed by the depletion of microglia, suggesting that microglia actively contribute to tau pathology during AD pathogenesis (Asai H, et al. Nat Neurosci 2015;18:1584). Activated microglia represent a common pathological feature of several neurodegenerative diseases, including AD, where microglial proinflammatory activity is increased and the microglial-mediated clearance mechanisms are compromised. Microglia are perpetually engaged in interactions with the surrounding environment in the CNS (Sarlus H and Heneka M T, J Clin Invest 2017;127:3240). Thus, the diverse microglial reactions at different disease stages may open new avenues for therapeutic intervention by modifying proinflammatory activities.

Activated macrophages are key mediators of several autoimmune and inflammatory diseases, for example, rheumatoid arthritis, lupus, Crohn's disease, ulcerative colitis, and atherosclerosis, etc. These activated macrophages over-express folate receptor β (FRβ; Xia W, et al. 2009;113:438). The abundance of activated macrophages in RA joints correlate to the degree of articular inflammation (Paulos C M, et al. Adv Drug Deliv Rev 2004;56:1205).

### B-Cells, B-Cell Depletion, and Repopulation

Non-specific immunoglobulins (IgGs) generated by B-cells may target Aβ and interfere with plaque formation and the progression of the disease. Thus, B-cells could contribute to AD pathogenesis beyond merely producing IgGs.

B-cells could be included in the onset and progression of AD in humans, especially when the BBB is breached. Transgenic mouse studies provide evidence that the loss of mature B-cells alone is sufficient to markedly arrest the progression of AD (Kim K, et al. Nat Commun 2021;12:2185). Activated B-cells, such as B1a and B1b, increase and accumulate both in peripheral circulation and brain parenchyma, resulting in IgG deposits around Aβ plaques. The progression of AD is also dependent on B-cells. Further, therapeutic depletion of circulating B-cells by an anti-CD20/B220 antibody at the onset of AD eliminates B-cells, IgG deposits, and reduces the Aβ plaque burden and microglial activation in the brain, thus blocking the onset of AD. In one embodiment, a therapeutic intervention strategy is to target B-cells in the brain and peripheral blood with a B-cell depleting therapy. For example, depleting subsets of B-cells could be a potential therapeutic intervention strategy to slow or halt the onset and progression of AD in CU individuals and MCI patients.

B-cells are extensively implicated in the pathogenesis of several autoimmune diseases, such as rheumatoid arthritis (RA), multiple sclerosis, and systemic lupus erythematosus (SLE), through the production of autoantibodies, presentation of autoantigens to T cells, T cell activation, and cytokine production (Rahman A and Isenberg DA, N Engl J Med 2008;358:929). B-cell depleting therapies have been approved to treat B-cell malignancies such B-NHL, CLL, and autoimmune diseases such as rheumatoid arthritis and multiple sclerosis. Examples of rituximab biosimilars approved for use in the US or EU include: Riabni, Rixathon, Ruxience, and Truxima. Clinical trials or treatment protocols involving the use of anti-CD20 therapies in AD are currently unknown.

Clinical response to rituximab treatment in RA has been correlated to B-cell depletion by employing ultra-sensitive assays such as minimal residual disease-flow cytometry (MRD-FC). Earlier conventional flow cytometry studies were less sensitive and failed to show a correlation with clinical outcomes (Dass S, et al. Arth Rheum 2008;58:2993). Incomplete depletion of peripheral B-cells leads to poorer clinical outcomes in terms of the magnitude and duration of the response to treatment. Slow but complete depletion still results in worse outcomes than patients in whom depletion was rapid and complete. Patients who have difficulty achieving peripheral clearance may be more likely to have difficulty clearing other compartments and thus subsequent poorer response. Examples of such compartments include the brain in AD, the synovium in RA, and lymph nodes and bone marrow in B-cell malignancies such as B-NHL, CLL.

MRD-FC analyses in chronic lymphocytic leukemia (CLL), which is a B-cell malignancy, demonstrated that the greatest reduction in B-cell tumor load in bone marrow correlates with full clearance of peripheral blood which in turn correlates to excellent clinical response to alemtuzumab treatment (Moreton P, et al. J Clin Oncol 2005;23:2971).

B-cell depletion can be achieved by a variety of methods. One such approach is the use of targeted antibody therapies, such as rituximab which binds to CD20 antigen on B-cells. Antibodies that target CD19 or CD22 antigen can also be employed to deplete B-cells. A major mechanism of action by which rituximab exerts clinical and therapeutic response rate is ADCC, which is dependent on the functional polymorphisms in FcGR-3A and FcGR-2A (Weng W-K and R. Levy, J Clin Oncol 2003;21:3940).

A single course rituximab monotherapy leads to complete response in some subset(s) of patients, whereas repeated administrations, i.e., maintenance therapy, could achieve complete response in additional subset(s) of patients in RA and SLE (Vital E M, et al. Arth Rheum 2010;62:1273-9; Vital E M, et al. Arth Rheum 2011;63:3038-47). Such responder and non-responder subsets could be identified, for instance, by determining either FcGR-3A, -2A functional polymorphisms and/or measuring B-cell subsets by MRD-FC.

Additional B-cell depleting anti-CD20 therapies include ocrelizumab, obinutuzumab, ofatumumab, and ublituximab, etc.

Highly sensitive analytical methods such as minimal residual disease-flow cytometry (MRD-FC) are used to measure subsets of B-cells. See, for example, in Dass S, et al. Arth Rheum. 2008;58:2993; Vital E M, et al. Arth Rheum 2011;63:603. Such a quantitation may be useful in determining when and how to treat an AD subject. The initial B-cell population parameters prior to initiating treatment will be useful in determining the effectiveness of rituximab, adalimumab, or lecanemab in AD subjects.

The functional polymorphisms in FcG receptors-3A and -2A could be determined by DNA, RNA, or protein sequencing. Each subject is assigned to one of the nine polymorphism groups in the 3×3 matrix.

MRD-FC can be performed throughout the treatment protocol, for example, immediately prior to administration of therapy, on day 15 before the second infusion of rituximab, 1 month after the second infusion, and at 3-month intervals thereafter. Maximum sensitivity can be achieved by incorporating the following features: (a) analysis of 500,000 events in each test; (b) enumeration of each B-cell subset over 2 separate tests, using the core markers phycoerythrin (PE)-Cy5.5-conjugated CD19, PE-Cy7-conjugated CD38, and allophycocyanin (APC)-labeled CD27, and light scatter characteristics to identify the primary B-cell subsets, as follows: naive=CD19+CD27−CD38+/−, memory=CD19+CD27+CD38+/−, and plasma cell precursors=CD19+(weak)CD27++CD38++; (c) incorporation and use of the additional markers fluorescein isothiocyanate (FITC)-labeled CD3 or CD24, PE-labeled CD86 or CD14, and APC-Cy7-conjugated CD45 or CD20 to confirm the phenotype and exclude contamination (for example, only CD19+(weak)CD27++CD38++ events that were also CD86+CD24− were classified as plasma cell precursors in the first test, while only CD19+(weak)CD27++CD38++ events that were also CD3−CD14−CD20− were classified as plasma cell precursors in the second test); (d) in the gating strategy of each test, inclusion of a marker that would not be expressed by the population of interest, in order to minimize contamination by events with nonspecific binding; (e) design and use of an extensive sequential gating strategy; this template was used for all patients, maximizing the ability of operators to recognize small variations in protein expression profile, thereby reducing any artifacts; (f) requirement for <25% coefficient of variation on the absolute total B-cell levels, and naïve, memory, and plasma cell precursor levels between the two tests; and (g) in each case, analysis of the results by two separate operators at the same facility. The limit of detection of the assay is typically defined as 0.002%, i.e., at least 10 events that satisfied all criteria in both tests or an absolute count of 0.0001×10⁹/liter (100 per ml), for a total white cell count of 5×10⁹/liter.

### ADCC and ADCP

The terms “antibody-dependent cell-mediated cytotoxicity” or “ADCC” are terms well understood in the art and refer to a cell-mediated reaction in which non-specific cytotoxic cells that express Fc gamma receptors (FcGRs) recognize bound antibody on a target cell leading to ADCC, for instance, cell killing. Non-specific cytotoxic cells that mediate ADCC include natural killer (NK) cells.

The term “antibody-dependent cell-mediated phagocytosis” or “ADCP” refers to a cell-mediated phagocytosis of an immune complex through an FcGR-mediated process. In AD, plaque clearance is via phagocytosis; an anti-Aβ antibody binds to the plaques and through Fc-mediated process the complex is engulfed by microglia. Examples of AD biologics that use the ADCP mechanism include: aducanumab, donanemab, gantenerumab, and lecanemab. Other immune cells that mediate phagocytosis include macrophages, monocytes, and neutrophils.

In some instances, the terms ADCC and ADCP are used interchangeably.

As used herein, the term “ADCC-dependent antibody therapy” refers to a therapy involving the use of an antibody that comprises an antigen-binding domain and an Fc region that binds to an Fc gamma receptor (FcGR) of a cytotoxic effector cells, for instance, macrophages in blood and microglia in the brain, where binding of the antibody to a target cell results in the killing of the target cell via ADCC. This killing of the target cell(s) provides a therapeutic effect in an individual. Additionally, binding of the therapeutic antibody-Aβ complex to FcGRs on microglia results in clearance of the Aβ plaques via ADCP. Examples of ADCP-dependent antibody therapies include aducanumab, donanemab, lecanemab, and gantenerumab, whereas examples of ADCC-dependent antibody therapies include adalimumab, infliximab, rituximab, and obinutuzumab.

An “ADCC-treatable disease or disorder,” as used herein, is a disease, condition, or disorder that is treated with a therapeutic antibody that mediates ADCC as a key mechanism of action (U.S. Pat. No. 8,592,149). Examples comprise neoplastic diseases, autoimmune diseases, inflammatory disorders, neuroinflammatory diseases, rare diseases, microbial infections, and allograft rejection. AD can be characterized as an ADCC-treatable disease or an ADCP-treatable disease depending on the mechanism of action of an antibody therapy.

Activated, proinflammatory macrophages in peripheral blood can be a therapeutic modality to treat AD. Macrophages over-expressing folate receptor beta (FRβ) can be depleted by administering an ADCC-mediating, anti-FRβ antibody (Feng Y et al. Arth. Res. Therap. 2011;13:R59).

### Fc-Gamma Receptors

The Fc receptors, members of the immunoglobulin gene superfamily of proteins, are surface glycoproteins that bind the Fc portion of immunoglobulins. Each member of the family recognizes immunoglobulins of one or more isotypes through a recognition domain on the gamma (γ) chain of the Fc receptor. Fc receptors are defined by their specificity for immunoglobulin subtypes. Fc receptors for IgG are referred to as FcGR (FcγR), for IgE as FεR, and for IgA as FcαR. Different accessory cells bear Fc receptors for antibodies of different isotypes, and the isotype of the antibody determines which accessory cells will be engaged given an immune response (Nimmerjahn F and J V Ravetch, Nat Rev Immunol. 2008;8:34). FcGRs, designated FcGRI (CD64), FcGRII (CD32), and FcGRIII (CD16), are encoded by distinct genes although they share extensive sequence homology.

FcGR-2(CD 32) is a 40 KDa integral membrane glycoprotein which binds complexed IgG; and exhibits only low affinity for monomeric Ig (appr. 10⁶ M⁻¹). FcGR-2 is the most widely expressed FcGR, present on all hematopoietic cells, including monocytes, macrophages including microglia, B cells, NK cells, neutrophils, mast cells, and platelets (Cohen-Solal et al. Immunol. Lett. 2004;92:199). FcGR-2 has only two immunoglobulin-like regions in its immunoglobulin binding chain and hence a much lower affinity for IgG than FcGR-1. There are three human FcGR-2 genes (FcGR-2A, FcGR-2B, FcGR-2C), all of which bind IgG in aggregates or immune complexes. Distinct differences within the cytoplasmic domains of FcGR-2A and FcGR-2B create two functionally heterogeneous responses to receptor ligation. FcGR-2A initiates intracellular signaling leading to cell activation such as phagocytosis and respiratory burst, whereas FcGR-2B initiates inhibitory signals leading to the inhibition of B-cell activation.

FcGR-2A has a functional polymorphism at position 131. Patients with homozygous alleles HH¹³¹ achieve better therapeutic and clinical response to rituximab monotherapy in B-NHL (Weng W-K and R. Levy, J Clin Oncol 2003;21:3940).

FcGR-3 (CD16) is a Type-I membrane protein that exists as two isoforms: FcGR-3A and FcGR-3B. Both FcGR-3A and FcGR-3B are low affinity receptors. FcGR-3A, an activating receptor, is expressed on NK cells, macrophages including microglia, monocytes, and dendritic cells; FcGR-3B, an inhibitory form, is expressed on neutrophils. All FcGRs bind the same region on IgG Fc, yet with differing high (FcGR-1) and low (FcGR-2 and FcGR-3) affinities (Sondermann P, et al. J Mol Biol 2001;309:737).

FcGR-3A has a functional polymorphism at position 158. Patients with homozygous alleles VV¹⁵⁸ achieve better therapeutic and clinical response to rituximab monotherapy in B-NHL (Weng W-K and R. Levy, J Clin Oncol 2003;21:3940). Copy number variations (CNV) exist (Nagelkerke S Q, et al. Front Immunol 2019;10:2237).

Macrophages and microglia express both FcGR-3A and -2A receptors. Both receptors are functionally relevant and independently included in binding IgGs to mediate ADCC and ADCP mechanisms.

### Stratified Medicine, Precision Medicine, Theragnostics

Heterogeneity in the AD patient population can obscure the therapeutic effects and clinical endpoints in all-comers clinical trials and treatment protocols. A potential solution lies in precision medicine protocols, which offer actionable therapeutic guidance. Such guidance can determine an AD patient's responsiveness to specific therapies, like lecanemab alone or in combination with adalimumab, tailored to distinct patient subsets or on a patient-by-patient basis. This approach is desirable for several reasons; it helps avoid delays in transitioning to alternative treatments, minimizes exposure to adverse effects such as ARIA-E/H, and reduces unnecessary healthcare costs. Moreover, there is a growing interest in administering antibody therapies at doses that minimize ARIA side effects, particularly in subset(s) of AD patients identified through mechanistic stratification or evaluated on a patient-by-patient basis. Consequently, there is a keen interest in the clinical development of therapies in specific subsets of AD patients, developing antibodies and biologic therapies suited for mechanistically stratified patient subsets, and identifying responders and non-responders among patients to optimize treatment, payment, and reimbursement strategies effectively.

Pharmaceuticals, ranging from small molecule drugs to protein biologics and therapeutic antibodies, are designed to interact with specific biological targets but exhibit variable treatment responses in real-world settings. This variability is often attributed to genetic differences across populations, including functional polymorphisms, which can influence the drug's therapeutic properties. These genetic factors can alter the drug's biological target, its metabolism, and the mechanisms through which it exerts its therapeutic effects. Functional polymorphisms in key receptors can affect, among others, the biological target of the drug, drug metabolism, and/or the biological mechanisms by which the drug mediates its therapeutic effect. Thus, a drug may only be effective in subjects who have a particular genetic or protein variation, with some individuals potentially experiencing adverse side effects or toxicity. Notably, therapeutic antibodies developed for treating cancers, autoimmune diseases, inflammatory disorders, and rare diseases have shown that their efficacy can significantly vary among patients, exemplified by the variable response to rituximab in treating follicular lymphoma, underscoring the need for personalized treatment approaches.

ADCC, ADCP: Many therapeutic antibodies mediate, wholly or in part, antibody dependent cell-mediated cytotoxicity or phagocytosis (XXXX, ADCP) as a major mechanism of action. ADCC is a process of cell-mediated immunity in which effector cells of the immune system, such as natural killer (NK) cells, macrophages, microglia, neutrophils, and eosinophils, kill target cells that have been bound by specific antibodies. Destruction or killing of the target cell can also occur through phagocytosis; ADCC-mediated lysis; ADCC-mediated apoptosis; and trogocytosis (antibody-dependent cytotoxicity mediated by polymorphonuclear granulocytes). ADCC is triggered by the binding of the effector cells to the Fc (constant) portion of the bound antibody through Fc receptors, particularly the FcG receptors, present on the effector cells. As such, variations or functional polymorphisms in the Fc receptor can affect the affinity and binding properties that dictate therapeutic and clinical effectiveness of antibodies, provided that ADCC or ADCP is a major therapeutic mechanism of action. The clinical and therapeutic correlations between FcGRs and ADCC led to the use of FcGR-3A, -2A polymorphisms for selecting patients for ADCC-mediated antibody therapies, for example, U.S. Pat. No. 8,592,149, which is incorporated herein by reference. While therapeutic antibodies may employ multiple therapeutic mechanisms, for example, ADCC, blocking cell signaling, apoptosis, or neutralization of antigens, etc., some antibodies use ADCC or ADCP as a major therapeutic mechanism and can be more discernable in monotherapy settings. In such settings, other mechanisms could contribute to the therapeutic effect albeit marginally.

Pursuant to, for example, 21 U.S.C. §321(g), the applicable regulations for diagnostic articles can be analogous to those for drugs, particularly “medicaments.” Under the description of “devices,” a component, part, or accessory of the article are similarly treated. In some cases, a diagnostic can be performed in a subject. In some embodiments, a diagnostic can be an assay. An assay can be carried out on a biological sample obtained from a subject. The diagnostic may be to evaluate baseline, to determine standards, to measure control levels, to evaluate rates of change, and other such measures. The diagnostic may cover specific methods, devices, or components within a measuring device. Non-limiting examples of the biological sample may include saliva, blood, serum, CSF, plasma, urine, a suspension of cells, or a suspension of cells and viruses. A biological sample may contain whole cells, lysed cells, plasma, red blood cells, skin cells, proteins, nucleic acids (for example, DNA, RNA, maternal DNA, maternal RNA), circulating nucleic acids (for example, cell-free nucleic acids, cell-free DNA/cfDNA, cell-free RNA/cfRNA), circulating tumor DNA/ctDNA, cell-free fetal DNA/cffDNA). As used herein, the term “cell-free” refers to the condition of the nucleic acid sequence as it appeared in the body before the sample is obtained from the body. For example, circulating cell-free nucleic acid sequences in a sample may have originated as cell-free nucleic acid sequences circulating in the bloodstream of the human body. In contrast, nucleic acid sequences that are extracted from a solid tissue, such as a biopsy, are generally not considered to be “cell-free.” In some cases, cell-free DNA may comprise fetal DNA, maternal DNA, or a combination thereof. In some cases, cell-free DNA may comprise DNA fragments released into a blood plasma. In some cases, the cell-free DNA may comprise circulating tumor DNA. In some cases, cell-free DNA may comprise circulating DNA indicative of a tissue origin, a disease or a condition. A cell-free nucleic acid sequence may be isolated from a blood sample. A cell-free nucleic acid sequence may be isolated from a plasma sample. A cell-free nucleic acid sequence may comprise a complementary DNA (cDNA). In some cases, one or more cDNAs may form a cDNA library.

In some cases, an assay can include a binding assay. As used herein, a “binding assay” can include a method used to determine the amount of binding of a component of a subject sample with a probe. Methods can include analytic biochemical methods such as electrophoresis, capillary electrophoresis, high performance liquid chromatography (HPLC), mass spectrometry including matrix-assisted laser desorption mass spectrometry (MALDI-MS), MALDI-time of flight mass spectrometry (MALDI-TOF), spectrophotometry, and the like. Direct binding can be measured using techniques such as an immunoassay. Examples of immunoassays include immunoprecipitation, particle immunoassays, immunonephelometry, radioimmunoassays, Western blot analysis, enzyme immunoassays (for example, ELISA), fluorescent immunoassays, and chemiluminescent immunoassays.

In some cases, a diagnostic can be a theragnostic. The term “theragnostic” encompasses products, tests, methods, and procedures that inform and guide the treatment of individual patients or groups of patients, such as subsets of subjects or entire populations affected by a specific disease like AD, under the coverage of payers, employers, or healthcare insurance companies. The primary objective of theragnostics is to significantly enhance treatment outcome within a reasonable timeframe, potentially reducing it from years to 3-6 months. These outcomes may range from disease remission and cure to excellent response rates and reduced disease progression. Theragnostic procedures are instrumental in generating critical clinical parameters specific to therapies, treatments, and treatment guidance. They could guide clinical trial designs, treatment protocols, and provide efficacy and financial assurances. Such assurances could be extended to payers, employers, and patients alike by various entities, including drug manufacturers, diagnostic companies, pharmacy benefit management (PBM) companies, or healthcare providers, aiming to optimize treatment efficiency and cost-effectiveness.

Theragnostic results can be applied to: (a) develop clinical trial design, including the selection of subjects or specific subsets of subjects, and the selection of drug candidates in trials; (b) guide prior authorization decisions for specialty drug use; (c) offer efficacy and financial assurances to payers and patients; (c) design a drug formulary, potentially a disease-specific formulary, where inclusion or exclusion decisions are based on theragnostic results. Theragnostic products can influence drug selection, guiding towards treatments based on the drug's mechanism of action and anticipated remission or excellent responses like halting or reversing the AD progression in a patient or subsets of patients. Drugs with moderate to poor efficacy may be altogether excluded from the formulary. Theragnostic methods can be mechanistic, focusing on: (a) the primary mechanism of action (MOA) of a drug and its efficacy in patients with specific genetic and/or immune profiles (for example, the MOA of lecanemab and donanemab being ADCP); and (b) the pathology of AD, categorized by specific immunological disease subtypes (for example, A⁺T⁻N⁻; A⁺T⁺N⁻; A⁺T⁺N⁺), alongside considerations of disease severity, pharmacology, B-cell subsets (naïve, memory, plasma B-cells as measured by MRD-FC), levels of inflammatory macrophages, levels of cytokines such as TNFα, IL-1, IL-6, etc., and clinical staging of AD (for example, CU, SCD, MCI, AD dementia), etc. Enhancing the precision of theragnostic procedures includes integrating one or more mechanistic determinants like ADCP, ADCC, ARIA, CAA, etc. For example, a comprehensive AD-specific theragnostic profile could be developed using a combination of stratification platforms, such as the 3 x3 matrix based on FcGR-3 A VF¹⁵⁸ and FcGR-2A HR¹³¹ functional polymorphisms, alongside immunological assays, ATN signature, biomarkers, diagnostic tests.

In a simpler embodiment, theragnostic procedures can provide treatment guidance to achieve preferred outcomes. This includes: (a) therapeutic appropriateness, that is, from among alternatives, directing specific drug selection for defined ranges of theragnostic readouts; (b) therapeutic guidance, that is, directing treatment schedule, from among alternatives, tailored drug dosing and treatment schedule for defined ranges of theragnostic readouts; (c) therapeutic effectiveness, that is, directing overall therapy strategy, from among alternatives, for defined ranges of theragnostic readouts; and (d) selection of alternative therapeutic, that is, specific exclusion criteria, from among alternatives, for particular other theragnostic readouts where treatment strategy may be contraindicated (for instance, by toxicity or side effects such as ARIA-E, -H) or if the first treatment strategy fails. These guidelines, rooted in theragnostic assessments, could become integral to drug (product) labeling, package inserts, or standalone diagnostic guidelines. Such protocols are essential for payers to authorize treatments and for specialty pharmacies to manage drug dispensation effectively.

A single or a combination of DNA, RNA, protein, or immunological features may constitute a theragnostic product or evaluation. In addition, it may include metabolic evaluation, which may be useful for individualized pharmacology of half-life, absorption, distribution, metabolism, excretion, turnover, and the like. Such examples can include biomarkers, polymorphisms, gene expression profiles, protein expression profiles, presence or absence of specific protein markers or immunological, metabolic or physiological profiles, and many aspects which affect the therapy response. Furthermore, a single or a combination of companion diagnostic tests or in vitro diagnostic tests (for example, theragnostics, complementary diagnostics) may constitute a theragnostic procedure.

Current biomarker, diagnostic tests, or companion diagnostic tests have limitations in fully realizing personalized medicine approaches in treatment settings: (a) lack of efficacy assurance (for example, guaranteed remission or superior response); (b) absence of financial assurance; (c) they are not employed for providing assurance-based treatment and payment decisions; (d) they are not utilized for the design and development of a drug formulary; or (e) they are not applied for facilitating disease-specific, population-wide therapy decisions, including the selection of a therapeutic alternative when the initially chosen drug does not achieve the anticipated therapeutic outcome.

CAA and ARIA Immune Mechanisms: In AD patients, intracerebral antigen-antibody immune complex (IC) formation induces inflammation in the brain that depends on Fc receptor interaction. In the presence of an antigen such as an Aβ plaque, non-specific autoantibodies can lead to a localized IC-mediated inflammatory reaction in the brain parenchyma and indirectly induce neuronal tissue damage through infiltration of leukocytes, and recruitment and activation of microglia via FcG receptors (Teeling J L, et al. Acta Neuropathol 2012;124:479). Additionally, the formation of ICs between Aβ peptides and non-specific IgGs, and subsequent FcGR-dependent inflammation can be a contributing factor in increased cerebral amyloid angiopathy (CAA), a condition in which amyloid deposits gradually replace the smooth muscle of blood vessel walls. Approximately half of the AD patients can have CAA.

While anti-Aβ antibody therapies facilitate the removal of plaques, they can also contribute to detrimental inflammatory reactions through interaction with perivascular macrophages and microglia. Amyloid-related imaging abnormalities (ARIA) can be observed in the brain via magnetic resonance imaging (MRI) in two radiographically distinct presentations, ARIA-E and ARIA-H, following the treatment of AD with anti-Aβ antibody therapies.

ARIA-E refers to vasogenic edema in the brain parenchyma or sulcal effusions (Sperling R A, et al. Alzheimers Dement 2011;7:367). ARIA-H refers to hemosiderin deposits presenting as microhemorrhages (hemorrhages<10 mm) in the brain parenchyma and/or localized superficial siderosis located in the subarachnoid space (Barakos J, et al. Am J Neuroradiol 2013;34:1958). The first reports of cerebral vasogenic edema and microhemorrhages associated with an anti-Aβ antibody therapy were observed following bapineuzumab treatment in a phase 2 multiple ascending dose trial (Salloway S, et al. Neurol 2009;73:2061). ARIA has also been reported in clinical trials of several other anti-Aβ antibody therapies, including gantenerumab, donanemab, lecanemab, and aducanumab.

The underlying mechanisms of ARIA have not been fully elucidated in clinical trials. In one embodiment, ARIA can be driven by antibody-mediated breakdown of accumulated Aβ aggregates in the cerebral blood vessels and brain parenchyma. This breakdown of cerebrovascular Aβ plaques may affect the structural integrity of the vessel wall, leading to increased permeability and leakage of proteinaceous fluid into surrounding tissues (Greenberg S M, et al. Nat Rev Neurol 2020;16:30). Additionally, mobilization of parenchymal Aβ plaques could impair perivascular clearance and/or initiate an immune response contributing to perivascular inflammation (Sperling R, et al. Lancet Neurol 2012;11:241). Preclinical studies have shown that there may be a recovery of vessel wall integrity and an increase in effective perivascular drainage with continued treatment, and, therefore, some clinical trials have observed that the risk of ARIA is highest early in treatment and subsequently decreases in patients who continue treatment (Sperling R, et al. Lancet Neurol 2012;11:241; Greenberg S M, et al. Nat Rev Neurol 2020;16:30).

ARIA-E may also occur spontaneously in patients with AD, presumably reflecting the inflammatory component of CAA (Carlson C, et al. Alzheimers Dement 2011;7:396). Similarly, both microhemorrhages and superficial siderosis are known to spontaneously occur in patients with CAA, independent of anti-Aβ antibody treatment (Goos J D, et al. Neurol 2010;74:1954).

In aducanumab Phase-3 clinical trials, overall, 41 percent of subjects taking the highest dose developed ARIA, compared to 10 percent of placebo controls. The ARIA incidence was dose-dependent. At 10 mg/kg, the dosage approved for clinical use, 35 percent of participants developed ARIA-E compared to 2.7 percent on placebo. Notably, 10 percent of subjects in the high-dose group had recurring ARIA-E episodes. About 40 percent of subjects with ARIA-E also had ARIA-H. About 6 percent of the high-dose cohort discontinued treatment due to ARIA-E or -H (Budd Haeberlein S, et al. J. Prev. Alzheimers Dis. 2022;9:197).

In lecanemab Phase-3 trial, 12.5 percent of subjects in the lecanemab group and 1.7 percent in the placebo group had ARIA-E. The symptomatic ARIA-E incidence was 2.8 percent in the lecanemab group and 0.0 percent in the placebo group. The ARIA-H rate was 17.0 percent in the lecanemab group and 8.7 percent in the placebo group. The symptomatic incidence of ARIA-H was 0.7 percent in the lecanemab group and 0.2 percent in the placebo group. The total incidence of ARIA (ARIA-E and/or ARIA-H) was 21.3 percent in the lecanemab group and 9.3 percent in the placebo group (van Dyck C H, et al. N Engl J Med 2023,388:9).

Severity or progression of AD could be influenced by immune mechanisms, for instance, ARIA and CAA. This may include activation of immune cells of formation of immune complexes at the sites of disease activity, a process mediated by FcG receptors.

The present disclosure provides methods, reagents, and kits for prognosing ARIA and/or CAA mechanisms. The methods, reagents, and kits find a number of uses, including, among others, selecting treatment options, treating AD subjects, and selecting AD subjects for particular treatments. Stratifying patients based on ARIA or CAA is desirable for tailored dosing and treatment schedule, selection of appropriate therapies, avoidance of delays in alternative treatments, reducing exposure to adverse effects of therapeutics, and reduction of unnecessary treatment expenses. The terms ARIA and CAA are used interchangeably, herein.

The present invention is based, in part, on the effects of functional polymorphisms of FcG receptors, particularly FcGR-2A, FcGR-3A, and FcGR-3B, on binding of IgG autoantibodies or therapeutic antibodies to the receptors. FcGR functional polymorphisms can be categorized into either low- or high-responder variants, wherein the high responder variants bind to IgG autoantibodies and/or IgG immune complexes (ICs) with high affinity. In AD, increased affinity and binding of IgG autoantibody-FcGR interactions with these high-binder variants likely leads to attraction of immune cells and deposition of ICs at disease compartments such as the brain. This activation of immune cells can exacerbate proinflammatory processes and lead to higher incidence of CAA or ARIA. Thus, a way of prognosing ARIA or CAA incidence is described herein based on the functional polymorphisms in FcGRs. In some embodiments, stratifying based on multiple FcGR functional polymorphisms, for example, two or more, allows a better identification of ARIA or CAA incidence.

Accordingly, in one aspect, the present disclosure provides a method of prognosing ARIA and CAA incidence and severity, the method comprising:

determining a polymorphism of an AD subject for one or more FcGR functional polymorphisms, wherein the FcGR-2A functional polymorphism is H/R¹³¹, and an FcGR-3A V/F¹⁵⁸ functional polymorphism; and prognosing the ARIA or CAA incidence and severity in the subject based on the determined polymorphism.

In some embodiments, the prognosis of AD severity is based on a combination of FcGR-2A H/R¹³¹ and FcGR-3A V/F¹⁵⁸polymorphisms, wherein (a) FcGR-2A H/H¹³¹, FcGR-3A V/V^{158 158} polymorphism is predictive of very high CAA and ARIA incidence; (b) a FcGR-2A H/H¹³¹, FcGR-3A V/F¹⁵⁸polymorphism; a FcGR-2A H/H¹³¹, FcGR-3A F/F¹⁵⁸ polymorphism; a FcGR-2A H/R¹³¹, FcGR-3A V/V¹⁵⁸, or a FcGR-2A R/R¹³¹, FcGR-3A V/V¹⁵⁸ polymorphism is predictive of high CAA and ARIA incidence; (c) FcGR-2A H/R¹³¹, FcGR-3A V/F¹⁵⁸ polymorphism is predictive of moderate CAA and ARIA incidence; (d) FcGR-2A H/R¹³¹, FcGR-3A F/F¹⁵⁸ polymorphism; or FcGR-2A R/R¹³¹, FcGR-3A V/F¹⁵⁸ polymorphism is predictive of low CAA and ARIA incidence; and (e) FcGR-2A R/R¹³¹, FcGR-3A F/F¹⁵⁸ polymorphism is predictive of very low CAA and ARIA incidence and severity.

In some embodiments, other prognostic indicators of CAA and ARIA incidence and severity can be used with the FcGR functional polymorphisms. These include, among others, the expression levels of FcG receptors, particularly on immune effector cells, such as peripheral neutrophils, monocytes, macrophages; and levels or change in levels of IgG autoantibodies in peripheral blood; ex vivo functional assays. In some embodiments, the ATN signature can be measured in stratified subsets based on the FcGR-3A/2A polymorphisms.

In some embodiments, the present disclosure also provides methods of assessing the CAA and ARIA incidence in AD. In some embodiments, the method comprises measuring the levels and/or change in levels of proinflammatory cytokines or chemokines, disease effector cells in peripheral blood (for example, neutrophils, monocytes, and macrophages, etc.), or combinations thereof. Measures of the CAA and ARIA can be an independent measure of disease severity and/or disease progression or used in combination with the polymorphism information of FcGR polymorphisms for a more refined prognosis of CAA and ARIA incidence and/or AD disease progression.

In another aspect, the prognosis of CAA or ARIA severity is applied to methods of selecting treatment options. In some embodiments, a method of selecting a treatment option, for instance, donanemab, for treating an AD subject comprises:
prognosing or stratifying CAA and ARIA incidence or disease progression in an AD subject;
and selecting or excluding a treatment option based on the prognosed ARIA incidence in the subject.

In some embodiments, the AD treatment option is selected from differential dosing, mono-and a combination therapy, differential dosing schedule, differential dosing frequency, and combinations thereof.

### DEFINITIONS

The terminology used herein is for the purpose of describing specific cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, for example, the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where specific values are described in the application and claims, unless otherwise stated the term “about” meaning within an acceptable error range for the value should be assumed. Also, where ranges and/or subranges of values are provided, the ranges and/or subranges can include the endpoints of the ranges and/or subranges.

As used herein, the terms “treatment,” “treating,” and the like, refer to obtaining a desired pharmacological and/or clinical effect. The effect may be prophylactic in terms of completely or partially preventing AD or symptom thereof and/or may be therapeutic in terms of a partial or complete cure for a disease and/or adverse effect attributable to the disease. “Treatment,” as used herein, covers any treatment of a disease in a human, and includes: (a) increasing survival time; (b) decreasing the risk of death due to the disease; (c) slowing, halting, or reversing the disease; (d) slowing, halting, or preventing the disease progression and/or disease severity; (c) preventing the disease from occurring in a subject which may be predisposed to the disease but has not yet been diagnosed as having it; (d) inhibiting the disease, i.e., arresting its development, for example, reducing the rate of disease progression; (e) halting the disease; (f) reversing the disease, for instance, causing regression of the disease, and (g) remission.

The terms “individual,” “host,” “subject,” and “patient,” used interchangeably herein, refer to a human.

“AD” refers to Alzheimer's Disease. It is a slowly progressing irreversible neurodegenerative disease with a long preclinical phase (up to 20+ years) and an average clinical phase of 8-10 years. The progression of AD is accompanied by changes to the brain that serve as biomarkers of the disease. Disease progression typically spans several stages, including preclinical AD, the MCI stage, and the AD dementia stage, which is comprised of mild, moderate, and severe dementia stages.

“CU” refers to the cognitively unimpaired stage in which AD patients do not exhibit any clinical symptoms. It precedes the MCI phase in the AD continuum. This stage can span 10-30 years before MCI stage can set in. Patients in the CU stage are cognitively normal (CN). A subset of CU individuals may have subjective cognitive impairment (SCD). CU and/or SCD subjects may have A⁺T⁻N⁻ signature (Cullen N C, et al. Nat Commun. 2021;12:3555).

CU individuals with a low burden of Aβ pathology, for instance, well before the presence of overt Aβ deposition in the brain, already show typical AD pathophysiological changes. Specifically, the main AD CSF biomarkers, reflecting tau, synaptic, and neurodegenerative changes, are already altered at this stage, while structural and functional brain changes are still minimally intact or absent. Plasma p-tau231 and p-tau217 better capture the earliest cerebral Aβ changes, before overt Aβ plaque pathology sets in, and are promising blood biomarkers to enrich a preclinical population for AD clinical trials (Cullen NC, et al. Nat Commun. 2021;12:3555; Milà-Alomà M, et al. Nat Med. 2022;28:1797) and treatment.

The Aβ plaque formation and accumulation trigger the clinical onset and further progression of AD through the deposition of tau tangles and NfL leading to neurodegeneration. Per a revised biological definition of AD, regardless of the presence of tau tangles (T⁻) or neurodegeneration (N⁻), the presence of Aβ (A⁺) alone puts a patient in the AD continuum (Jack C R Jr, et al. Alzheimers Dement. 2018;4:535). Based on this hypothesis, the therapeutic intervention strategy is to find ways for faster and complete clearance of Aβ plaques and soluble aggregates which can then potentially improve cognition and/or daily function, and possibly stop or even reverse the progression of AD.

“MCI” refers to mild cognitive impairment in AD. Also referred to as prodromal stage of AD, it is the first clinically symptomatic stage of AD, in which there is evidence of AD pathology and impairment in one or more cognitive functions, but symptoms do not interfere with daily functioning. It is an early stage of memory loss in the AD continuum if the hallmark changes in the brain are present. Amnestic MCI primarily affects memory while non-amnestic MCI affects thinking skills other than memory, including the ability to make sound decisions, judge the time or sequence of steps needed to complete a complex task, or visual perception. Individuals living with MCI who have an abnormal PET scan, CSF, or plasma Aβ markers can be considered to have early AD.

“Dementia” in AD refers to decline of cognitive function in an AD patient, and it is the last stage in AD continuum. Dementia can be further staged into mild, moderate, and severe depending on the extent of loss of cognitive and/or functional abilities.

“Early AD” refers to MCI and/or mild AD dementia stages.

“ATN signature” refers to the presence or absence of Aβ, tau protein, and NfL protein in plasma, CFS, or the brain. Studies on combinations of blood-based Aβ42/Aβ40, P-tau, and NfL biomarkers have demonstrated their diagnostic accuracy for the separation of AD dementia from CU individuals and patients with non-AD disorders.

The usefulness of plasma Aβ42/Aβ40, P-tau217, and NfL, either separately or in combination, was evaluated for predicting longitudinal cognitive decline and clinical outcomes in a CU population comprised of both cognitively normal (CN) healthy controls and individuals with subjective cognitive decline (SCD) who did not meet the diagnostic criteria for MCI. The ATN plasma biomarkers could improve the prediction of cognitive decline and risk of developing AD dementia compared to using only basic demographic information. This diagnostic strategy could reduce the sample size required to conduct AD clinical trials in CU elderly population by 60-70% (Cullen N C, et al. Nat Commun. 2021;12:3555).

“CDR-SB” refers to Clinical Dementia Rating-Sum of Boxes, which is a validated outcome measure used in clinical trials of AD obtained by interviewing patients and their care partners to analyze cognitive and functional abilities. It assesses six domains that patients and caregivers identify as important (memory, orientation, judgment and problem solving, community affairs, home and hobbies, and personal care). Scores for each domain range from 0 to 3, with higher scores indicating greater impairment. Total scores range from 0 to 18, with a score of 0.5 to 6 indicating early AD.

“ADAS-Cog14” refers to The Alzheimer's Disease Assessment Scale-Cognitive Subscale that assesses the level of cognitive dysfunction in AD. ADAS-Cog is the most common cognitive assessment instrument used in AD clinical trials. ADAS-Cog 14 consists of 14 competencies: word recall, commands, constructional praxis, object and finger naming, ideational praxis, orientation, word recognition, remembering word recognition instructions, comprehension of spoken language, word finding difficulty, spoken language ability, delayed word recall, number cancellation, and maze task. ADAS-Cog scale has been used in clinical trials for earlier stages of AD including MCI.

“ADCOMS” refers to AD Composite Score, which consists of 4 Alzheimer's Disease Assessment Scale-cognitive subscale items, 2 Mini-Mental State Examination items, and all 6 CDR-SB items. ADCOMS demonstrates improved sensitivity to clinical decline over individual scales in prodromal AD (pAD), amnestic MCI (aMCI), and in mild AD dementia. Improved sensitivity predicts smaller sample size requirements when ADCOMS is used in early AD trials (Wang J, et al. J Neurol Neurosurgery & Psychiatry 2016;87:993-9).

“Monotherapy” refers to the administration of a single drug to treat a disease. A drug can be administered as a single course or maintenance therapy in monotherapy.

“Combination therapy” refers to the administration of two or more drugs to treat a disease. A combination therapy can be administered as a single course or maintenance therapy. Routes of administration can vary, for instance, one drug may be administered subcutaneously and the other intravenously. The drug combination can include a biologic and a pill. The term combination product is used interchangeably to refer a combination therapy.

A combination product is a product composed of any combination of a drug and a device, where the device may include a diagnostic or theragnostic test; a biological product and a device; a drug and a biological product; or a drug, device, and a biological product. Under 21 CFR 3.2 (e), a combination product is defined to include:

A product comprised of two or more regulated components (i.e., drug/device, biologic/device, drug/biologic, or drug/device/biologic) that are physically, chemically, or otherwise combined or mixed and produced as a single entity, which is often referred to as a “single entity” combination product;

Two or more separate products packaged together in a single package or as a unit and comprised of drug and device products, device and biological products, or biological and drug products, which is often referred to as a “co-packaged” combination product;

A drug, device, or biological product packaged separately that according to its investigational plan or proposed labeling is intended for use only with an approved individually specified drug, device, or biological product where both are required to achieve the intended use, indication, or effect and where, upon approval of the proposed product, the labeling of the approved product would need to be changed (for example, to reflect a change in intended use, indication, dosage form, strength, route of administration, or significant change in dose), which is often referred to as a “cross-labeled” combination product; or Any investigational drug, device, or biological product packaged separately that according to its proposed labeling is for use only with another individually specified investigational drug, device, or biological product where both are required to achieve the intended use, indication, or effect, which is another type of “cross-labeled” combination product.

“Single course” refers to a one-time treatment, for instance, a single time or multiple times of administration of a drug administered within a short period of time. For instance, antibody therapy, for instance, rituximab monotherapy, can be administered in four, weekly infusions within a month, collectively referred to as a single course therapy (Weng W-K and R. Levy, J Clin Oncol 2003;21:3940). Similarly, an antibiotic treatment administered as daily or twice daily pills over five days is considered as a single course treatment.

“Maintenance therapy” refers to a therapy, therapeutic regimen, or a course of therapy administered several times, for instance, on a weekly, biweekly, or monthly basis for an extended period, for instance, six months to two years. In some instances, the treatment schedule can comprise an induction therapy, for instance, an initial course of therapy administered to a subject to halt or reverse the progression of the disease or disorder, followed by one or more administrations of the therapy to maintain the improvement in health achieved by the induction therapy, or “consolidate” the gains obtained by the induction therapy. Administration of anti-Aβ therapies or anti-TNF therapies on a weekly, biweekly, or monthly basis for six months or more is considered as a maintenance therapy.

“Critical clinical parameters” refers herein to disease-specific, theragnostic or diagnostic parameters that are necessary to determine mechanisms of action of drugs, incidence of ARIA or CAA mechanism, disease subtypes, or a combination thereof to determine treatment or clinical response in a subject.

“Responsiveness” in a subject refers to a treatment outcome or a clinical outcome of a treatment or therapy for a disease or disorder. The treatment outcome or clinical outcome can be measured according to standards recognized in the art for specific diseases such as AD and RA. Surrogate endpoint such as the clearance of Aβ plaques can also be a measure of treatment outcome.

“Predicting” refers to determining the probability or likelihood of a particular outcome or event. In reference to responsiveness to treatment, the term refers to the likelihood of a particular treatment outcome or clinical outcome.

“Predicting responsiveness”, or “providing a prognosis” or “prognosing”, refers to predicting whether the antibody maintenance therapy will have an impact on disease progression.

“Functional polymorphism” refers to the single nucleotide polymorphisms at the IgG binding site in FcGR-3A and -2A receptors. In FcGR-3A it is the valine/phenylalanine amino acid change at position 158, whereas in FcGR-2A it is the histidine/arginine amino acid change at position 131. The Fc portion of an IgG 1 therapeutic antibody, autoantibody, or immune complex binds to the FcG receptors and mediates mechanisms such as ADCC, ADCP, ARIA, or CAA. These polymorphisms dictate clinical, therapeutic, and immunes responses. NA1/NA2 is the functional polymorphism in FcGR-3B receptor.

“APOE4” allele refers to an isoform of ApoE. It is a major genetic risk factor for AD in a gene dose-dependent manner increasing risk by up to 15-fold in homozygotes. APOE4 contains arginine residues at 112 and 158 positions.

“Stratifying” or “stratification” refers to classifying subjects into distinct groups or strata based common characteristic(s) or trait(s). Stratification can be based on a single trait or two or more traits, for instance, FcGR-3A, -2A, and -3B functional polymorphisms.

“Reference stratification” as used herein refers to an established stratification scheme that has stratified a treatment response/clinical outcome-polymorphism association, with statistically significant differences between the different groups in the stratification. Accordingly, a subject having an ADCC- or ADCP-treatable disease whose polymorphism for the FcG receptor polymorphism (for example, FcGR-2A and/or FcGR-3A) is known and can be compared to the reference stratification to identify the likelihood of the subject having a particular treatment outcome or clinical outcome, i.e., responsiveness, for an antibody maintenance therapy.

“Correlating,” “correlation,” “correlates,” as used herein refer to the establishment of a relationship, for example, mutual or reciprocal, between polymorphism status and therapeutic and/or clinical efficacy of certain treatments as described herein. Correlating refers to relating the polymorphism status to responsiveness to treatment or therapy.

“Excluding a treatment or therapy” refers to removing a possible treatment from consideration, for example, for use on a particular patient or subsets of patients, for instance, based on the presence or absence of a particular variance(s) in one or more genes or immune markers in that patient.

“Excluding a subject” refers to removing the subject from consideration of a treatment or therapy, including in reference to treatment or therapy in clinical trials, for instance, based on the presence or absence of a particular variance(s) in one or more genes or immune markers in that patient.

“Selecting a treatment or therapy” refers to including a possible treatment for consideration, for example, for treating a particular patient based on the presence or absence of a particular variance(s) in one or more genes of that patient.

“Selecting a subject” refers to including the subject for consideration of a treatment or therapy, including in reference to treatment or therapy in clinical trials, based on the presence or absence of a particular variance(s) in one or more genes of that patient.

“Autoimmune disease or disorder” refers to a disease state that is immune mediated against self-tissues, external stimuli, microbial pathogens, and/or substances normally present in the body. It is generally associated with production of inflammatory factors, which further promote tissue destruction. Inflammatory microglia trigger AD pathology. Inflammatory macrophages, inflammatory NKT cells, etc., can cause chronic inflammatory diseases such as atherosclerosis, Type-2 diabetes, sickle cell disease. Autoimmune diseases can be systemic or organ specific. Examples of systemic autoimmune diseases include Alzheimer's disease, multiple sclerosis, rheumatoid arthritis, systemic lupus erythematosus, ankylosing spondylitis, scleroderma and Sjogren's syndrome. Examples of organ-specific autoimmune diseases include Alzheimer's disease, autoimmune hemolytic anemia, Goodpasture's syndrome, Grave's disease, Hashimoto's thyroiditis, idiopathic thrombocytopenia purpura, insulin-dependent diabetes mellitus, myasthenia gravis, pernicious anemia, lupus nephritis, and psoriasis.

“Inflammatory disease” refers to a disease or disorder caused by or resulting from or resulting in inflammation. The term “inflammatory disease” may also refer to a dysregulated inflammatory reaction that causes an exaggerated response by macrophages, granulocytes, and/or T-lymphocytes leading to abnormal tissue damage and cell death. In some embodiments, an inflammatory disease or disorder can be an aspect of other diseases, such as autoimmune diseases.

AD can be classified as an autoimmune disease, an inflammatory disorder, or a neuroinflammatory disorder.

“Anti-CD20 antibody” refers to an antibody that specifically binds to cell surface protein CD20, which is expressed on certain cells of the immune system, specifically the B-lymphocyte differentiation antigen Bp35. As used herein “CD20” includes, for example, variants, isoforms, and species homologs of human CD20. Anti-CD20 antibodies may, in certain instances, cross-react with CD20 of more than one species. In some embodiments, the antibodies may be completely specific for one or more human CD20 proteins and may not exhibit species or other types of non-human cross-reactivity. Exemplary anti-CD20 antibodies include rituximab, ocrelizumab, obinutuzumab, ofatumumab, veltuzumab, ublituximab, etc. Anti-CD20 antibodies are also described in U.S. Pat. Nos. 6,399,061 and 7,422,739. Responders to anti-CD 20 therapies tend to effectively deplete B-cells or subsets of B-cells. Currently, B-cell depleting therapies including anti-CD20 antibody therapies are not approved for the treatment of AD.

“Minimal residual disease” (MRD) is a term used to describe a very small number of cells, for example, B-cells, macrophages, other immune cells, cancer cells, etc. MRD can be found only by highly sensitive detection methods, for example, MRD-flow cytometry (MRD-FC), that are able to find ultra-low or rare number of cells. For example, the limit of detection is typically defined as an absolute count of 0.0001×10⁹/liter (100 per ml), for a total white cell count of 5×10⁹/liter.

“Target cell depletion assay” refers to an ADCC or ADCP assay measuring the reduction, depletion or killing of cells targeted by an antibody. Target cell depletion assay can be done in vitro, for instance, a subject's B-cells are used ex vivo with an anti-CD20 antibody and effector cells. In some embodiments, the target cell depletion assay can be done in vivo, for example, by measuring number of B cells in a subject after the administration of an anti-CD20 antibody by withdrawing blood samples and measuring time-dependent depletion assays over a period of several weeks or months. Typically, MRD-FC is one way to measure these populations in peripheral blood. Such depletion studies can include depletion of proinflammatory macrophages by an anti-TNF therapy, for instance, adalimumab.

“Target cell repopulation assay” refers to an ADCC assay measuring the slow or faster recovery rates of a target cell population following administration of an antibody therapy, for instance, repopulation of B cells. Repopulation assays can be done in vivo, for instance, by measuring specific subsets of B cells such as naïve, memory, and plasma subsets repopulating after the administration of an anti-CD20 antibody by withdrawing blood samples over a period of several weeks to months. Typically, MRD-FC is used to longitudinally measure these populations in vitro. Faster repopulation of subset(s) of B-cells, for instance, memory B-cells, is a measure of imminent disease relapse.

“ARIA” refers to amyloid-related imaging abnormalities (ARIA) observed in the brain via magnetic resonance imaging (MRI) in two radiographically distinct presentations, ARIA-E and ARIA-H, following the treatment of AD with anti-Aβ antibody therapies. ARIA-E refers to vasogenic edema in the brain parenchyma or sulcal effusions. ARIA-H refers to hemosiderin deposits presenting as microhemorrhages (hemorrhages<10 mm) in the brain parenchyma and/or localized superficial siderosis in the subarachnoid space.

“CAA” refers to cerebral amyloid angiopathy, which is a form of naturally occurring ARIA-E, -H inflammation in the brain.

“Clinical trial” refers to an investigation of safety and/or efficacy of a treatment for a disease or disorder. Typically, clinical trials are carried out to obtain approval from a governmental regulatory agency for marketing a drug. Besides conventional Phase-1, 2, and 3 trials, the term herein can include P-4 trials, RWE trials, 3^{rd} party-sponsored trials, payer-participating trials, N=1 trials, and N=1 efficacy trials.

The term “N=1 efficacy” herein refers to achieving therapeutic efficacy, clinical efficacy, or financial value in a treated patient. Such efficacy can include different cut off scores, for example, remission, cure, slowing the disease progression, etc. In one embodiment, sizeable trials may provide N=1 efficacy on a subject-by-subject basis. Irrespective of the size of a sizeable clinical trial, in N=1 efficacy trial, efficacy outcome is specific to that subject in the trial or treatment protocol. Sizeable trial refers to ultra-large trials; a maximum of 1 million patients per disease or per drug can participate in the trial.

The term “N=1 assurance” herein refers to providing patient-specific efficacy assurance and/or financial assurance for a patient participating in a trial or treatment protocol.

The term “assurance” herein refers to a form of guarantee, for example, therapeutic guarantee, product guarantee, product performance guarantee, value guarantee, service guarantee, financial guarantee, and the like. Assurance can also be referred to herein as reinsurance. Assurance can limit the amount of loss a third-party payer, for example, Medicare or an employer, or a patient can incur.

An “insurer” refers to relationships where an entity has a contractual obligation to provide medical care to a patient. The plan may approve or limit treatment of a patient with a particular drug, for instance, by way of prior authorization. Historically and traditionally, insurers are not third-party payers of clinical trials, for example, sizeable trials.

The term “third-party payer” or “payer” herein refers to an entity that funds a clinical trial, for example, a sizeable clinical trial of N=10000, 30000, 100000, or 1000000 or more per disease and/or per drug, and bears a substantial component of the clinical trial costs including medicament and/or treatment costs. A patient may bear a portion of the drug cost, for example, coinsurance amount if it is a specialty drug. In some instance, a patient may pay a component of the clinical trial costs, for example, about 3% of the per-patient clinical trial cost, about 7% of the per-patient clinical trial cost, or in excess of about 20%, for example, about 30%, 40%, 50%, 60%, 70%, 80%, 90%, etc., can be paid. Such trials are herein referred to as ‘pay-to-participate’ trials. In some embodiments, a pharmaceutical company that developed, manufactured, or commercialized the drug is not a third-party payer. A third-party payer can be a private insurance company, a government entity (for example, a government healthcare entity such as U.S. Medicare system, UK PHS), a private payer, an employer or a consortium of employers, a pension fund, and the like. A payer also may encompass a managed care company. In one embodiment, a fully-integrated payer may provide additional functions that can be offered by a managed care company, for example, pharmacy benefit management (PBM), specialty pharmacy, disease and therapy management, theragnostic evaluation, and the like. In some embodiments, a fully-integrated payer can be a non-profit managed care entity, for example, Kaiser Permanente. In some embodiments, such a managed care company may also provide integrated clinical trial capabilities and/or healthcare provider capabilities. In another embodiment, such a managed care company, through vertical or lateral integration with a large pharmaceutical company, may provide integrated clinical trial capabilities and/or healthcare provider capabilities. Traditionally, clinical trials are funded by either pharmaceutical companies (who are also the clinical trial sponsor) or successors of interest. Historically, traditional sponsors have not conducted such sizeable trials.

In some embodiments, trials of significantly larger size than the typical, limited, patients-funded, pay-to-participate trials, are envisioned. In particular, trials of larger size, distinguishable as “sizeable” from earlier “limited” trials of about 70 subjects (N=70), for example, about 80, 90, 100, 110, 120, 130, 140, 150, 170, 190, 210, etc., can be included. In one embodiment, a sizeable trial may include 30,000 subjects. In one embodiment, a sizeable trial may include 100,000 subjects. In one embodiment, a sizeable trial may include 1,000,000 subjects in an AD trial. In some embodiments, this can include trials comparing, for example, biosimilar as alternative to a reference biologic, including a specialty drug or a reference drug listed in the FDA Orange, Purple Book, or a foreign counterpart thereof. In various such sizeable trials, larger percentages of participants have clinical trial costs paid by such third-party payers, for example, in excess of about 70%, for example, about 75, 80, 85, 90, 95, 97, 99, etc. In some cases, patients enrolled in the trial pay for drug costs and/or trial costs, for example, 3% of the per-patient trial cost, or in excess of about 30%, for example, about 40%, 50%, 60%, 70%, 80%, 90%, etc. See PCT/US2019/018811.

The terms “third-party clinical trial sponsor” or “sponsor” or “clinical trial provider” herein refers to an entity that conducts a clinical trial. In one embodiment, a managed care company sponsors a sizeable clinical trial and may employ telehealth architecture to conduct such sizeable clinical trials, for example, single-site, virtual, or site-less clinical trials. In embodiments, a pharmaceutical company that developed, manufactured, or commercialized the drug is not a sponsor, does not fund the trial, nor will it have direct access to the clinical trial data. In some embodiments, a fully-integrated third-party payer, for example, a payer with managed care capabilities, or a managed care company can be a sponsor. Such a sponsor is responsible for initiating, managing, funding, and/or otherwise ensuring that the clinical trial is properly performed, complying with safety, ethical, statistical, reproducibility, and other aspects of a clinical trial. In some embodiments, a fully-integrated provider can be a non-profit managed care entity, for example, Kaiser Permanente. Traditionally, a sponsor may include a pharmaceutical company, a patient advocacy foundation, or an academic clinical investigator included in investigator-initiated trials, for example, limited trials. In some embodiments, a sponsor can be an entity which tests a biosimilar or generic counterpart or equivalent to a reference biologic or small molecule drug, for example, including a specialty drug or a reference drug listed in the FDA Orange, Purple Book, or a foreign counterpart thereof.

The term “clinical trial merger” herein refers to a context wherein a clinical trial and treatment paradigms are seamlessly overlapping. In one embodiment, all treated patients in a treatment protocol participate in a clinical trial, wherein clinical trial grade data are collected, for instance, for the purposes of development and submission of data to a regulatory agency. In some embodiments, said clinical trial is a phase-2 clinical trial, phase-3 clinical trial, or a phase-4 clinical trial. In some embodiments, said phase-3 clinical trial is wholly funded by a third-party payer. In some embodiments, said phase-4 clinical trial is wholly funded by a third-party payer. In some embodiments, said phase-3 or -4 clinical trials are conducted by a third-party sponsor, for example, a managed care company. In some embodiments, said clinical trials are sizeable trials, that is, larger than limited trials. In some embodiments, said phase-4 clinical trial tests an FDA-approved drug in an approved disease indication, and that trial employs 40000 subjects insured by a third-party payer. Such a clinical trial merger meets two objectives: (a) a sizeable clinical trial is conducted to generate necessary clinical data, for example, long-term disease remission and disease relapse patterns, excellent versus worst responder subsets, N=1 efficacy; and (b) third-party payer can simultaneously administer drugs to its patients.

The term “medicament” can be used to refer to an approved drug or a drug candidate that is undergoing a clinical trial. A “drug” can include any therapeutic that has completed a regulatory approval process (for example, FDA, or a foreign counterpart) in a given indication, for instance, adalimumab in RA, and lecanemab in AD. A “drug candidate” can include any therapeutic that has not completed a regulatory approval process in a given indication, for instance, adalimumab in AD. In some cases, a drug or a drug candidate can be a therapeutic that may be undergoing, or has undergone a Phase I, Phase II, Phase III, or a Phase IV clinical trial as defined by the FDA. In some cases, a drug candidate can be a therapeutic that has not undergone a Phase I, Phase II, Phase III, or a Phase IV clinical trial as defined by the FDA. These terms may also encompass various diagnostic methods, theragnostic products, medical devices, cell-based therapies and therapeutics, nucleic acids-based therapies and therapeutics, and the like.

The term “label revision” refers to modifications made to an existing drug label that has been approved by a regulatory agency, such as the FDA. Such revisions may include issuing a new label for treating a condition not previously covered by the drug's approval, targeting treatment to a specific subset of patients rather than an all-comers population, introducing a new route of administration, requiring the inclusion of a companion diagnostic for selecting appropriate patients or patient subsets for treatment, adjusting the dosage, etc. The term as used herein is intended to encompass related concepts, including label restriction, label expansion, or the issuance of a completely new label.

The term “real-world data” (RWD)refers to data relating to patient health status and/or the delivery of healthcare routinely collected from a variety of sources. Examples of RWD can include data derived from electronic health records (EHRs); medical claims data; data from product and disease registries; patient-generated data, including from in-home-use settings; and data gathered from other sources that can inform on health status, such as mobile devices. The term “real-world evidence” (RWE) refers to the clinical evidence about the usage and potential benefits or risks of a medical product derived from RWD analysis (see, for example, Framework for FDA's real-world evidence program, December 2018, www.fda.gov).

The term “synthetic control arm” or “SCA” includes data collected from subjects enrolled in conventional treatment platform, for example, real-world data that has previously been collected from sources such as health data generated during routine care, including electronic health records; administrative claims data; patient-generated data from fitness trackers or home medical equipment; disease registries; historical clinical trial data; and the like. By reducing or eliminating the need to enroll control participants, a SCA can increase efficiency, reduce delays, lower trial costs, and accelerate life-saving therapies to market. For instance, instead of having to recruit 1,000 patients, for example, 500 for the active arm, and 500 for the control arm, only 500 participants need to be recruited in the trial when a SCA is employed. SCA is also referred to as external control arm, which uses data collected from outside of the current trial to provide a comparator group and are especially useful in N=1 trials and for studies of rare disease treatments where it is not feasible or ethical to collect data from randomized trials.

The term “specialty drug” can include a drug that is prescribed to treat a complex, chronic, rare, or difficult-to-manage disease or disorder. In some cases, such a disease can be a neuroinflammatory disease, an autoimmune disease, an inflammatory disorder, a cancer, a rare disease, and the like (PCT/US2019/018811; US 20190378597; US 20210225475). Most drugs developed through biotechnology processes are specialty drugs. The term “protein” can be used interchangeably to encompass both naturally-and non-naturally occurring polypeptides, and fragments, mutants, derivatives, and analogs thereof. A protein may be monomeric or polymeric. Further, a protein may comprise several different domains each of which has one or more distinct activities. In some cases, a protein can be at least 40 amino acids in length. A protein can comprise an overall charge based on pKa of side chains of component amino acids. In some instances, a protein can have an overall positive charge. In some instances, a protein can have an overall negative charge. In some instances, a protein can have an overall neutral charge. A protein can furthermore exist as a zwitterion. In some cases, the charge of the side chains may depend upon the local conditions of evaluation, for example, solution pH.

The term “recombinant” refers to a biomolecule, for example, a gene or protein, that (a) can be removed from its naturally occurring environment, (b) can be isolated from all or a portion of a polynucleotide in which the gene may be found in nature, (c) can be operatively linked to a polynucleotide which it may not be linked to in nature, or (d) may not occur in nature” The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. Thus, for example, a protein synthesized by a microorganism can be recombinant, for example, if it is synthesized from an mRNA synthesized from a recombinant gene present in the cell.

The terms “administration,” “co-administration,” “administered in combination with” and their grammatical equivalents or the like, as used herein, can encompass administration of medicaments to a subject, and can include treatment regimens in which medicaments are administered by the same or different routes of administration, or at the same or different times. In some embodiments, a medicament can be co-administered with other agents (for example, other medicaments). These terms can encompass administration of one or more medicaments to a subject so that medicaments and/or their metabolites are present in the subject at the same time. They can include simultaneous administration, administration at different times, and/or administration in a composition in which one or more medicaments are present. Thus, in some embodiments, a medicament(s) can be administered in a single composition. In some embodiments, a medicament(s) can be admixed in the composition. In some embodiments, the same medicament can be administered via a combination of different routes of administration. In some embodiments, medicament(s) administered can be in a therapeutically effective amount. In some embodiments, prodrugs may be converted into active forms.

A “regulatory agency” can include a drug regulating authority that is responsible for enforcing rules and regulations, and issuing guidelines for drug development, licensing, registration, manufacturing, marketing, and labeling of pharmaceutical, diagnostic, combination products, etc. Examples of regulatory agencies can include the US Food and Drug Administration (FDA); the UK Medicines and Healthcare Products Regulatory Agency (MHRA); the Australian Therapeutic Goods Administration (TGA); the Indian Central Drug Standard Control Organization (CDSCO); Health Canada; the European Medicines Agency (EMEA); the Danish Medicines Agency; the Swedish Medical Products Agency (MPA); the German Federal Institute for Drugs and Medical Devices; the Swiss Agency for Therapeutic Products; and the Japanese Ministry of Health, Labour & Welfare (MHLW).

### GENERAL

Products such as medicaments, diagnostics, and databases resulting from a clinical trial may require regulatory clearance by agencies like the FDA. However, not all products from a clinical trial necessarily require regulatory clearance.

The costs for participating in a clinical trial are typically covered by a clinical trial sponsor. Unlike conventional or classical trials which are often sponsored by drug manufacturers, the trials discussed here could be sponsored by entities without direct ownership or licenses to the medicament's intellectual property. Additionally, in these trials, a third-party payer might only cover the costs if the treatment results in successful outcomes, for instance, clinical remission. In some embodiments, such non-classical trials can be treatment-cum-trial protocols, wherein every treated patient with a licensed and approved drug is in clinical trial by generating longitudinal, clinical trial-grade data.

A clinical trial sponsor may use both licensed and unlicensed or non-approved drugs in clinical trials. Unlicensed medicaments, including biosimilars or generics of approved drugs, can be tested under patent exemptions. This approach allows for the development and submission of clinical data to regulatory bodies, aiming to secure approval for the medicament's use.

Clinical trials might operate under the auspices of the medicament's manufacturer or independently, led by clinical trial providers or third-party sponsors. In some cases, the roles of sponsor and provide overlap. Funding can also come from governmental research entities, like the NIH or NCI, or be supported by third-party payers such as insurers or pension funds, covering various costs associated with the trial.

In certain scenarios, regulatory bodies have authorized a clinical trial provider or third-party sponsor to charge for medicaments used in trials. Examples of such regulatory agencies include the Food and Drug Administration (FDA), the European Medicines Agency (EMEA), the Indian Central Drug Standard Control Organization (CDSCO), and the Japanese Ministry of Health, Labour & Welfare (MHLW).

Clinical trials often include the administration of medicaments, with some trials administering a single medicament, while others may administer multiple. These medicaments can be administered based on various paradigms, such as sequentially or in combination. Furthermore, medicaments may be administered using the same or different routes, including injections or infusions (e.g., subcutaneous, intravenous, perispinal, intrathecal, or intracranial delivery). To address the limitations of intravenously delivered biologic therapies, ultra-high concentrated, subcutaneous self-administered formulations can be employed. The administration paradigm for one medicament in a trial might or might not align with that of another medicament within the same trial.

Clinical trial data, once collected, may be entered into a specifically designed database system. This database could contain a variety of data types, including but not limited to clinical trial data, diagnostic data collected during the trial, and possibly theragnostic data. It is important to note that the database might host data not directly derived from the clinical trial itself. The inclusion of data within this database is flexible, accommodating both data generated within the trial and relevant external data, enhancing the scope and utility of the database for comprehensive analysis.

Clinical trials are structured to incorporate appropriate controls to ensure their integrity and reliability of the results. These controls may encompass strategies like subject randomization, use of placebos, use of double blinds. Furthermore, modern trials may employ innovative approaches such as synthetic control arms and digital twins to enhance study outcomes. While these elements are commonly expected, not all trials will incorporate every one of these controls. The nature of a clinical trial could range from a randomized clinical trial (RCT) to an adaptive clinical trial (ACT), and it may be designed as either prospective or retrospective, with or without double-blinding. Occasionally, a study might diverge from these standard models, utilizing a retrospective analysis of a disease registry instead.

Following a clinical trial's successful conclusion, the next phase often includes targeted marketing efforts for the investigated medicament. These efforts are designed to inform and persuade healthcare providers, such as physicians, nurses, and hospitals, as well as insurance providers, about the benefits of the new therapy. Marketing strategies might include educational programs, publication of study results in peer-review journals and various advertising media. There is also a focus on patient-directed marketing, which might employ channels like print media, television, and the internet to reach individuals diagnosed with conditions such as AD, highlighting patient-specific and targeted therapy options.

### EXAMPLES

### Example-1: Non-Classical AD Trial in Rheumatoid Arthritis Patients

It is a treatment-cum-trial protocol sponsored by a third-party. Both the drug and trial costs are covered by a third-party payer, for instance, Medicare. Patients also have the option to pay for the drug and treatment costs, for example, 20% of the costs in the form of coinsurance amount. Drug developers commercializing these drugs do not sponsor this trial.

Anti-Aβ therapies offer limited clinical and therapeutic benefits in certain patient subsets. These treatments are not effective in all treated patients and do not necessarily stop the progression of AD in all treated MCI and early AD patients.

The trial aims to identify suitable therapies to potentially slow, stop, or reverse the early stages of AD, particularly in CU or early MCI patients who have also been diagnosed with an autoimmune disease such as RA, denoted as RA_{CU}; RA_{MCI} patients.

A subset of CU individuals presents subjective cognitive decline symptoms (SCD; Cullen N C, et al. Nat Commun. 2021;12:3555) with an A⁺T⁻N⁻ signature. SCD participants, not meeting MCI criteria, display significantly lower baseline Mini-Mental State Examination (MMSE, P<0.001) and Preclinical Alzheimer's Cognitive Composite (PACC, P<0.001) scores, a markedly increased risk of AD dementia (P<0.001), and elevated baseline plasma NfL levels (P=0.04). These patients are targeted for recruitment in this trial.

The trial investigates whether FDA-approved drugs for RA might be effective for AD. Lecanemab is approved for treating AD, while donanemab could be approved soon. In addition to microglia-mediated Aβ plaque clearance in the brain, this trial examines whether peripheral blood macrophages can also effectively clear Aβ plaques.

Adalimumab, etanercept, and rituximab, while marketed for rheumatoid arthritis, have not been approved for AD treatment. The study explores the potential of anti-TNF therapies, such as adalimumab and etanercept, in targeting proinflammatory pathways, and examines the effectiveness of B-cell depleting therapy, specifically rituximab, in potentially modifying the course of early-stage AD.

The drugs are purchased at cost by the trial sponsor. The pharmaceutical companies commercializing the drugs are not included in the trial's execution or access to its data.

All treated patients or specific subset(s) within the protocol participate in this clinical trial, with the aim of assessing the drug's efficacy on a patient-by-patient basis, i.e., N=1 efficacy. The protocol encompasses 100,000 participants. See Ching E P and Ramakrishnan V, PCT/US2019/018811.

This comprises a multi-arm, adaptive protocol featuring FDA-approved therapies. See Woodcock J and Lavange L M, New Engl J Med 2017;377:62-70.

For additional details into clinical trial deigns, primary and secondary endpoints in AD, and methods for conducting such trials, refer to Woodcock J and Lavange L M, New Engl J Med 2017;377:62; Butchart J, et al. Neurol 2015;84:2161; and Mintun M A, et al. New Engl J Med 2021;384:1691.

The protocol can be characterized as an in-house, RWE trial as it integrates treatment and clinical trial, facilitated by a single third-party sponsor through an integrated platform.

Patients from the early RA pool, within 1-2 years of diagnosis, are selected based on plasma ATN signature criteria, for example, A⁺T⁻N⁻, A⁺T⁺N⁻. Baseline assessments of B-cell subsets and PET imaging data are made for patient selection.

The trial is conducted by further stratifying patient populations based on the therapeutic mechanism(s) of action of the drugs, i.e., ADCC and ADCP. FcGR-2A, and -3A polymorphisms are determined to generate the 3×3 matrix leading to nine groups, where Group-1 is characterized by FcGR-2A HH¹³¹ and FcGR-3A VV¹⁵⁸ polymorphisms, and Group-9 by FcGR-2A RR¹³¹and FcGR-3A FF¹⁵⁸ polymorphisms. Patients are further stratified based on FcGR-3B NA1/NA2 polymorphism. The APOE4 polymorphism is also measured.

Clinical endpoints include evaluating the impact on AD progression, aiming to slow, halt, or reverse disease progression, or to achieve clinical remission, in subjects undergoing either monotherapy or combination therapy. For SCD (CU) participants, primary endpoints are cognition or AD dementia.

Elderly RA patients with MCI or SCD are recruited. Baseline assessments include: Immunoglobulin G (IgG) rheumatoid factor (RF)^{+/-}, IgG anti citrullinated protein antibodies (ACPA)^{+/-}, Immunoglobulin A (IgA) RF^{+/-}, IgA ACPA^{+/-}, C-reactive protein (CRP)^{High/Low}, citrullinated fibrinogen (cFib)^{+/-}; and FcGR-3A and -2A polymorphisms. Further immune markers, including B-cell subsets, proinflammatory macrophages, cytokines such as TNFα, IL-1, IL-6 are longitudinally monitored at intervals, for instance, 3, 6, 9, 12, 15, and 18 months, to correlate these markers to clinical or therapeutic outcomes, cognitive decline, and CAA or ARIA incidence and severity.

Lecanemab is administered as monotherapy to subjects in FcGR subsets Groups-1,2,3,4, and 7. Additional monotherapy arms include adalimumab and rituximab.

The fourth arm is lecanemab and adalimumab combination therapy for subjects in FcGR subsets Groups-1,2,3,4, and 7. The fifth arm is lecanemab and rituximab combination therapy for the same subsets.

The sixth arm is lecanemab as monotherapy to subsets Groups-5,6,8, and 9, determined by FcGR-3A and -2A polymorphisms. Etanercept is explored as a monotherapy in a separate arm, with a subsequent arm testing lecanemab and etanercept combination therapy.

Subjects are treated with the following therapies for six months to two years. RA and AD clinical evaluations for every 3 months. Plasma levels of TNF-α and IL-6 levels, plasma ATN levels, depletion and re-population of B-cell subsets, inflammatory macrophages are measured just before the initiation of treatment and every three months thereafter.

50 mg etanercept monotherapy is administered subcutaneously once weekly. Adalimumab monotherapy is administered 40 mg once weekly subcutaneously. Leqembi dosage is 10 mg/kg, administered intravenously once every two weeks. Rituximab dosage is two-1000 mg intravenous infusions separated by two weeks (one course) every 24 weeks.

Cross-over provisions are made for non-responders to alternative therapeutic arms as needed.

Subjects undergoing combination therapies remain in CU or early MCI status for 2-3 years as evidenced by plasma ATN biomarkers, PET imaging, and cognitive and functional outcomes, effectively halting progression to AD dementia. Additionally, these subjects achieve excellent response in RA, as indicated by the ACR-70 score, within 6-12 months of treatment initiation. Follow-up in open-label extension studies demonstrated sustained CU or early MCI status for up to 5 years.

Subjects in monotherapy arms, such as those receiving adalimumab, rituximab, or etanercept, also show sustained CU or early MCI status for 2-3 years, corroborated by plasma ATN biomarkers, PET imaging, and cognitive and functional outcomes. RA symptoms were in remission within six months of treatment initiation. Notably, combination therapies involving lecanemab, for instance, in patient groups-1,2,3,4, and 7 with an AT-N-signature, demonstrate complete plaque depletion as determined by plasma ATN signatures and/or PET imaging, preventing progression to AD dementia for three years.

Trial findings are submitted to the FDA, seeking new drug labels for adalimumab, etanercept, and rituximab for treating CU (SCD) individuals and MCI patients. Additionally, a novel theragnostic label is generated to guide these mono-and combination therapies in defined subsets, aimed at decelerating or halting AD progression. The proposed labels mention the use of these therapies to mitigate AD progression and treat MCI patients, marking a new indication for these biologics previously unapproved for AD treatment. The data generated in this trial supports the establishment of a class label for biosimilars of adalimumab, extending the label's applicability beyond the reference drug to encompass all its biosimilars. Another example is, based on the results generated from the lecanemab trial, a class label is generated covering all ADCP-dependent anti-Aβ therapies including aducanumab and donanemab.

Payers, including Medicare (CMS) in the US and NHS-England, endorse these therapies for AD treatment, incorporating these drugs into drug formularies. Prior authorization by payers facilitates the dispensation of these therapies to patients for self-administration or to specialist physicians for intravenous delivery.

The theragnostic label mentions the use of adalimumab, etanercept, or rituximab as either monotherapy or in combination with lecanemab for treating CU and MCI patients, targeting specified subsets as delineated above.

### Example-2: Classical Phase-4 Clinical Trial in MCI and Early AD Patients

The trial is conducted to further investigate the therapeutic effectiveness of lecanemab in SCD (CU) individuals. See (van Dyck C H, et al. N Engl J Med 2023;388:9-21). This trial may be solely sponsored by the manufacturer or include a third party, such as a co-sponsor. Alternatively, a third-party payer may cover part of the drug or trial costs. The co-sponsor's role includes stratifying subjects based on FcGR-3A and -2A functional polymorphisms to enhance patient selection, trial design, and interpretability of results. ATN biomarkers from plasma samples and other immune markers described in Example-1 are assessed in stratified subsets or on a patient-by-patient basis. B-cell depletion and repopulation metrics are also evaluated. See Example-5.

3,000 subjects are enrolled, distributed across nine patient groups defined by the 3 x3 FcGR matrix. Plasma samples are analyzed to identify ATN signatures, and patients with A⁺T⁻N⁻ or A⁺T⁺N⁻ signatures are enrolled.

Over several months, lecanemab maintenance therapy leads to superior primary and secondary outcomes in Groups-1, 2, 3, 4, and 7, with a significant majority achieving AB plaque clearance within 6-9 months. Group-5 shows moderate responses, Groups-6, 8, and 9 exhibit poor responses. Superior outcomes include achieving a CDR-SB score improvement of 1.5 or more, either specific subsets or on a patient-by-patient basis.

An accelerated approval pathway yields a new product label for lecanemab monotherapy, aimed at treating SCD individuals to decelerate or prevent progression to MCI or AD dementia. This label might also specify criteria for selecting subsets or individuals for treatment. Concurrently, a new theragnostic label is developed to define the intent-to-treat populations, facilitating more targeted and effective treatments.

### Example-3: Classical Phase-2 Trial in MCI Patients

The objective of the trial is to determine if donanemab significantly improves CDR-SB scores in specific patient groups compared to what was observed in all-comers Phase-3 trial (van Dyck C H, et al. N Engl J Med 2023;388:9-21). Further, the clinical outcomes are assessed in a combination therapy setting.

This Phase-2b trial is randomized, multi-arm, placebo controlled, conducted by a third-party trial sponsor. Drug and trial costs are covered by the sponsor, excluding participation by the drug developer or payers.

The trial includes 600 MCI patients without additional autoimmune disease like RA. The trial spans two years, extends for two more years in an open-label setting.

Monotherapy arms include donanemab, adalimumab, and rituximab, within patient groups defined by FcGR functional polymorphisms (Groups-1, 2, 3, 4, and 7). The combination therapy arms include donanemab with adalimumab, donanemab with rituximab; and adalimumab with rituximab. Donanemab and rituximab are administered intravenously, while adalimumab is administered subcutaneously.

Refer to Example-1 for additional trial specifications. B-cell depletion and repopulation measurements are made by MRD-FC.

Significant outcomes are seen in both monotherapy and combination therapy groups, particularly in slowing AD dementia progression as indicated by CDR-SB scores.

A new product label is generated for donanemab monotherapy and its combination therapies, aimed at treating MCI patients in stratified subsets. Additionally, theragnostic label is generated to select treatment eligible subsets.

### Example-4: Classical Phase-2 Clinical Trial in SCD Individuals

A Phase-2(b) adaptive trial, randomized, placebo controlled, and double-blinded, is sponsored by an independent precision medicine company covering the drug and trial costs. Drug manufacturers and payers are not included.

Involving 600 SCD subjects over three years, with a two-year open-label follow-up, it excludes individuals with underlying autoimmune or inflammatory indications. Subjects are stratified based on FcGR-3A, -2A polymorphisms, and plasma ATN profiles, focusing on Groups-1, 2, 3, 4, and 7 with A+T-N signatures. The trial arms include rituximab and adalimumab monotherapies and a placebo arm. B-cell depletion and repopulation measurements are made by MRD-FC.

Significant outcomes were achieved in both adalimumab and rituximab arms. Over two years, about 40% remained in the SCD stage with A⁺T⁻N⁻signature, while 60% reverted to CU, free from SCD with A⁻T⁻N⁻ signature. In contrast, 70% of participants in the placebo arm advanced to MCI, with the remainder developing AD dementia.

Accelerated approvals lead to new indications for adalimumab and rituximab in treating SCD. Additionally, theragnostic label is generated to select treatment eligible subsets.

### Example-5: B-Cell Depletion and Repopulation Measurements in AD Trials

An analysis of B-cell depletion in AD trials including RWE trials is performed, analogous to European League Against Rheumatism (EULAR) outcomes in RA (Van Gestel, et al. Arth Rheum 1996;39:34-40). The outcome of rituximab maintenance therapy in AD can be correlated with the amount of B-cell depletion. The quantitation may be useful in determining when and how to treat an individual, for example, in an FcGR subset. Such a quantitation is also useful in determining complete versus partial depletion and/or rapid versus slow depletion of B-cells during the trial period.

In FcGR stratified patient groups or on a subject-by-subject basis, the clinical and therapeutic outcomes in AD are further correlated to B-cell depletion and repopulation patterns.

A study is performed to show that the outcome from antibody maintenance therapy, for example, rituximab monotherapy or lecanemab-rituximab combination therapy in AD can be correlated to the depletion and repopulation patterns of B-cells. The therapeutic outcomes of both the monotherapy and combination therapies correlate with the rates (fast versus slow) and extents (complete versus partial) of population depletion and subsequent repopulation patterns, for example, in Groups-1, 2, 3, 4, and 7.

Specific embodiments provided herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments so claimed are inherently or expressly described and enabled herein.

In cases where numerical values are indicated herein, the skilled person will understand that the technical effect of the feature in question is ensured within an interval of accuracy, which typically encompasses a deviation of the numerical value given of ±10% or of ±5%. At the very least, and not as an attempt to limit the application of the doctrine of equivalents to the scope of the claims, each numerical parameter should at least be construed considering the number of reported significant digits and by applying ordinary rounding techniques.

Unless otherwise indicated, all numbers expressing quantities of ingredients, properties such as molecular weight and median size, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained.

The terms “a,” “an,” “the” and similar referents used in the context of the description herein (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the specification and does not pose a limitation on the scope of the claims. No language in the specification should be construed as indicating any non-claimed element essential to the practice of the description.

Unless specifically stated or obvious from context, as used herein, the term “or” is understood to be inclusive and covers both “or” and “and.”

Groupings of alternative elements or embodiments provided herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments are described herein, including the best mode known for carrying out methods provided herein. Of course, variations on these described embodiments will become apparent upon reading the foregoing description. One can be expected to employ such variations as appropriate and can be practiced other than as specifically described herein. Accordingly, this description includes all modifications and equivalents of the subject matter recited in the claims appended hereto as permitted by applicable law. Moreover, any combination of the above-described elements in all possible variations thereof is encompassed by the description unless otherwise indicated herein or otherwise clearly contradicted by context.

It is to be understood that the embodiments provided herein are illustrative of the principles of the description herein. Other modifications that can be employed are within the scope of the description. Thus, by way of example, but not of limitation, alternative configurations can be utilized in accordance with the teachings herein. Accordingly, the presented information is not limited to that precisely as shown and described.

While the present description has been described and illustrated herein by references to various specific materials, procedures, and examples, it is understood that the description is not restricted to the particular combinations of materials and procedures selected for that purpose. Numerous variations of such details can be implied as will be appreciated. It is intended that the specification and examples be considered as exemplary only, with the true scope and spirit of the specification being indicated by the following claims. All references, patents, and patent applications referred to in this application are herein incorporated by reference in their entirety.

Unless defined otherwise, all technical and scientific terms used herein have the same meaning as commonly understood. Although other probes, compositions, methods, and kits similar, or equivalent, to those described herein can be used in the practice described herein, the materials and methods are described herein. It is to be understood that the terminology used herein is for the purpose of describing embodiments only and is not intended to be limiting.

Unless specifically stated or obvious from context, as used herein, the term “about” is understood as within a range of normal tolerance, for example within 2 standard deviations of the mean. About is understood to be within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

A stated range is understood to be any value between and at the limits of the stated range. As examples, a range between 1 and 5 includes 1, 2, 3, 4, and 5; a range between 1 and 10 includes 1, 2, 3, 4, 5, 6, 7, 8, 9, and 10; and a range between 1 and 100 includes 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100.

Any aspect or embodiment described herein can be combined with any other aspect or embodiment as described herein.

What is claimed is:

1. A method of conducting a clinical trial in Alzheimer's Disease (AD) subjects, comprising:
a) stratifying AD subjects based on a combination of therapeutic mechanisms of action and disease subtypes to make enrollment decisions;
b) administering to the stratified subsets or on a subject-by-subject basis, at least one of the following drug candidates:
i) an anti-beta-amyloid (anti-Ab) antibody,
ii) an anti-TNF biologic,
iii) a B-cell depleting antibody,
iv) a combination therapy consisting of an anti-Ab antibody and an anti-TNF biologic, or a combination therapy consisting of an anti-Ab antibody and a B-cell depleting antibody.

2. The method of claim 1, wherein the anti-Ab antibody is selected from the group consisting of lecanemab, donanemab, aducanumab, or gantenerumab.

3. The method of claim 1, wherein the anti-TNF biologic is selected from the group consisting of adalimumab, etanercept, or infliximab.

4. The method of claim 1, wherein the B-cell depleting antibody is an anti-CD20 antibody therapy, such as rituximab.

5. The method of claim 1, wherein the diagnostic tests further include imaging studies, such as magnetic resonance imaging (MRI) or positron emission tomography (PET) scans, to assess brain amyloid deposition or neurodegeneration.

6. The method of claim 1, wherein the clinical trial is selected from Phase-2, Phase-3, Phase-4, real-world evidence (RWE) trials, or N=1 efficacy trials.

7. The method of claim 1, wherein stratifying AD subjects includes:
i) determining a combination of therapeutic mechanisms of action and FcGR functional polymorphisms, wherein the polymorphisms include FcGR-3A V/F158 and FcGR-2A H/R131, wherein the therapeutic mechanisms include antibody dependent cellular cytotoxicity (ADCC) or antibody dependent cellular cytotoxicity (ADCP);
ii) correlating the incidence of amyloid-related imaging abnormalities (ARIA) or cerebral amyloid angiopathy (CAA) immune mechanism is correlated to the FcGR functional polymorphisms, wherein the polymorphisms include FcGR-3A V/F158 and FcGR-2A H/R131;
iii) measurement of biomarkers including Ab42/Ab40 ratio, phosphorylated-tau217 (P-tau217), and neurofilament light chain (NfL) in plasma or cerebrospinal fluid (CSF) to refine patient stratification and treatment selection;
iv) measurement of immune cells including B-cell subsets and inflammatory macrophages, wherein the B-cell subsets include naïve, memory, and plasma B-cell subsets; wherein the measurement includes depletion or repopulation of the immune cells in peripheral blood;
v) measurement of cytokines in peripheral blood, wherein the cytokines include TNF-a, IL-1, IL-6; and
vi) measurement of disease subtypes including from the group consisting of cognitively unimpaired (CU), subjective cognitive decline (SCD), mild cognitive impaired (MCI), or AD dementia states.

8. The method of claim 1, wherein the stratification based on functional polymorphisms further includes assessing additional genetic or immune markers that may influence and/or predict treatment response to the drug candidates.

9. The method of claim 1, wherein the stratified subsets or subjects are further categorized based on the severity of disease symptoms at the time of enrollment, such as mild, moderate, or severe AD.

10. The method of claim 1, wherein the combination therapy consisting of an anti-Ab antibody and an anti-TNF biologic is administered sequentially, with the anti-TNF biologic given prior to the anti-Ab antibody.

11. The method of claim 1, wherein the combination therapy consisting of an anti-Ab antibody and a B-cell depleting antibody includes concurrent administration to maximize treatment outcomes.

12. The method of claim 1, wherein the combination therapies are co-administered.

13. A clinical trial in AD subjects, the trial comprising:
a) stratifying AD subjects based on a combination of therapeutic mechanisms of action and disease subtypes to make enrollment decisions;
b) administering to the stratified subsets or on a subject-by-subject basis, at least one of the following drug candidates:
i) an anti-Ab antibody,
ii) an anti-TNF biologic,
iii) a B-cell depleting antibody,
iv) a combination therapy consisting of an anti-Ab antibody and an anti-TNF biologic, or a combination therapy consisting of an anti-Ab antibody and a B-cell depleting antibody.

14. The clinical trial of claim 13, the trial further comprising methods to stratify AD subjects including:
i) determining a combination of therapeutic mechanisms of action and FcGR functional polymorphisms, wherein the polymorphisms include FcGR-3A V/F158 and FcGR-2A H/R131, wherein the therapeutic mechanisms include antibody dependent cellular cytotoxicity (ADCC) or antibody dependent cellular cytotoxicity (ADCP);
ii) correlating the incidence of amyloid-related imaging abnormalities (ARIA) or cerebral amyloid angiopathy (CAA) immune mechanism is correlated to the FcGR functional polymorphisms, wherein the polymorphisms include FcGR-3A V/F158 and FcGR-2A H/R131;
iii) measurement of biomarkers including Ab42/Ab40 ratio, phosphorylated-tau217 (P-tau217), and neurofilament light chain (NfL) in plasma or cerebrospinal fluid (CSF) to refine patient stratification and treatment selection;
iv) measurement of immune cells including B-cell subsets and inflammatory macrophages, wherein the B-cell subsets include naïve, memory, and plasma B-cell subsets; wherein the measurement includes depletion or repopulation of the immune cells in peripheral blood;
v) measurement of cytokines in peripheral blood, wherein the cytokines include TNF-a, IL-1, IL-6; and
vi) measurement of disease subtypes including from the group consisting of cognitively unimpaired (CU), subjective cognitive decline (SCD), mild cognitive impaired (MCI), or AD dementia states.

15. A method of treating an AD patient using a precision medicine approach, comprising:
a) stratifying AD subjects includes:
i) determining a combination of therapeutic mechanisms of action, disease subtypes, ARIA/CAA immune mechanism, and FcGR functional polymorphisms, including FcGR-3A V/F158 and FcGR-2A H/R131 to make enrollment decisions;
ii) measurement of biomarkers including Ab42/Ab40 ratio, phosphorylated-tau217 (P-tau217), and neurofilament light chain (NfL) in plasma or cerebrospinal fluid (CSF) to refine patient stratification and treatment selection;
iii) measurement of immune cells including B-cell subsets and inflammatory macrophages, wherein the B-cell subsets include naïve, memory, and plasma B-cell subsets;
iv) measurement of cytokines in peripheral blood, wherein the cytokines include TNF-a, IL-1, IL-6;
b) administering at least one of the following treatments to the stratified subsets or on a patient-by-patient basis:
i) an anti-Ab antibody therapy,
ii) an anti-TNF therapy,
iii) a B-cell depleting antibody therapy,
iv) a combination therapy consisting of an anti-Ab antibody therapy and an anti-TNF therapy, or
v) a combination therapy consisting of an anti-Ab antibody therapy and a B-cell depleting therapy;
c) regularly monitoring the patient's cognitive and functional status using standardized outcome measures such as ADAS-Cog or CDR-SB scores to assess the effectiveness of the treatment and adjust the therapy as needed.

16. The method of claim 15, wherein the anti-Ab antibody is selected from the group consisting of lecanemab, donanemab, aducanumab, or gantenerumab.

17. The method of claim 15, wherein the anti-TNF biologic is selected from the group consisting of adalimumab, etanercept, or infliximab.

18. The method of claim 15, wherein the B-cell depleting antibody is an anti-CD20 antibody therapy, such as rituximab.

19. The method of claim 15, further comprising utilizing PET imaging and MRI scans to monitor changes in brain Ab deposition and neuroinflammation as part of the regular monitoring.

20. The method of claim 15, wherein the stratification based on the FcGR functional polymorphisms includes FcGR-3A V/F158 and FcGR-2A H/R131 polymorphisms.

21. The method of claim 15, further comprising the step of adjusting the treatment based on the patient's response to therapy, including modifying the dosage or switching to a different therapy within the prescribed options.

22. The method of claim 15, wherein the biomarker profiles include measuring plasma levels of Ab42/Ab40 ratio, phosphorylated-tau217, and neurofilament chain.

23. The method of claim 15, wherein the biomarker profiles include measuring depletion and/or repopulation of B-cell subsets, wherein the subsets include naïve, memory, and plasma B-cells, wherein the measuring includes minimal residual disease-flow cytometry (MRD-FC).

24. The method of claim 15, wherein the biomarker profiles include measuring depletion and/or repopulation of inflammatory macrophages, wherein the subsets include macrophages overexpressing folate receptor b.

25. The method of claim 15, wherein the biomarker profiles include measuring cytokines levels in peripheral blood, wherein the cytokines include TNF-a, IL-1, and IL-6.

26. The method of claim 15, further comprising the step of enrolling the patient in a patient registry or observational study to collect long-term efficacy and safety data on the precision medicine approach.

27. The method of claim 15, wherein the dosing or scheduling of the combination therapy is tailored based on the extent of ARIA/CAA incidence, wherein the incidence can be measured by the FcGR functional polymorphisms or the levels of AD-specific autoantibodies in peripheral blood.

28. A method of administering a drug candidate in an AD subject enrolled in a clinical trial, wherein the trial protocol comprises:
a) selecting the drug candidate based on stratification criteria that include functional polymorphisms, therapeutic mechanisms, ARIA/CAA incidence, disease subtypes, and biomarker profiles;
b) the drug candidate being one or more of an anti-Ab antibody, an anti-TNF biologic, or a B-cell depleting antibody, administered either as a monotherapy or part of a combination therapy;
c) evaluating treatment response by measuring changes in cognitive and functional status; and
d) adjusting the drug candidate based on these evaluations.

29. A method of conducting a clinical trial in AD subjects with an underlying autoimmune disease or inflammatory disease, comprising:
a) stratifying patients based on AD pathology, underlying autoimmune or inflammatory disease, and biomarkers indicative of disease subtypes;
b) administering a drug candidate either as a monotherapy or part of a combination therapy, wherein the monotherapy or combination therapy comprises at least one of the following:
i) an anti-Ab antibody, ii) an anti-TNF biologic, or iii) a B-cell depleting antibody; and
c) assessing the dual effectiveness of the drug candidates on AD progression and the underlying condition through predefined clinical endpoints and biomarker changes.

30. The method of claim 29, wherein the underlying autoimmune or inflammatory disease is selected from the group consisting of rheumatoid arthritis, psoriatic arthritis, Crohn's disease, ulcerative colitis, and inflammatory bowel disease, wherein the rheumatoid arthritis can be early or very early rheumatoid arthritis.

31. The method of claim 29, wherein the AD pathology is selected from the group consisting of cognitively unimpaired (CU), subjective cognitive decline (SCD), mild cognitive impaired (MCI), or AD dementia states.

32. A clinical trial in AD subjects with an underlying autoimmune disease or inflammatory disease, the trial comprising:
a) stratifying patients based on AD pathology, underlying autoimmune or inflammatory disease, and biomarkers indicative of disease subtypes;
b) administering a drug candidate either as a monotherapy or part of a combination therapy, wherein the monotherapy or combination therapy comprises at least one of the following:
i) an anti-Ab antibody,
ii) an anti-TNF biologic, or
iii) a B-cell depleting antibody; and
c) assessing the dual effectiveness of the drug candidates on AD progression and the underlying condition through predefined clinical endpoints and biomarker changes.

33. A method of stratifying AD patients for targeted treatment, comprising:
determining a combination of therapeutic mechanisms of action and FcGR functional polymorphisms, wherein the polymorphisms include FcGR-3A V/F158 and FcGR-2A H/R131, wherein the therapeutic mechanisms include antibody dependent cellular cytotoxicity (ADCC) or antibody dependent cellular cytotoxicity (ADCP);
b) correlating the incidence of amyloid-related imaging abnormalities (ARIA) or cerebral amyloid angiopathy (CAA) immune mechanism is correlated to the FcGR functional polymorphisms, wherein the polymorphisms include FcGR-3A V/F158 and FcGR-2A H/R131;
c) measurement of biomarkers including Ab42/Ab40 ratio, phosphorylated-tau217 (P-tau217), and neurofilament light chain (NfL) in plasma or cerebrospinal fluid (CSF) to refine patient stratification and treatment selection;
d) measurement of immune cells including B-cell subsets and inflammatory macrophages, wherein the B-cell subsets include naïve, memory, and plasma B-cell subsets; wherein the measurement includes depletion or repopulation of the immune cells in peripheral blood;
e) measurement of cytokines in peripheral blood, wherein the cytokines include TNF-a, IL-1, IL-6;
f) measurement of disease subtypes including from the group consisting of cognitively unimpaired (CU), subjective cognitive decline (SCD), mild cognitive impairment (MCI), or AD dementia state; and
g) selecting an appropriate therapy for each patient based on a combination of genetic and immune biomarkers, and clinical data, aiming to slow the progression of AD or improve cognitive and functional outcomes.

34. A method of treating an AD patient, the method comprising:
a) administering an anti-Ab antibody as a first therapy to the subject;
b) subsequent to or concurrent with step (a), administering a second therapy selected from the group consisting of an anti-TNF biologic and a B-cell depleting antibody;
c) determining the patient's responsiveness to the first and second therapy based on changes in at least one of cognitive function and functional status; and
d) adjusting the treatment regimen based on the determined responsiveness, wherein the adjustment comprises modifying the dose, frequency, or combination of the first therapy and second therapy to optimize therapeutic outcomes for the patient.

35. A method of conducting a classical clinical trial in an unstratified population of AD subjects, comprising:
a) enrolling subjects diagnosed with AD without prior stratification based on mechanism of action of therapies, mechanism of ARIA or CAA incidence;
b) randomly assigning the enrolled subjects to receive at least one therapy selected from the group consisting of:
i) an anti-Ab antibody as a monotherapy,
ii) an anti-TNF biologic as a monotherapy,
iii) a B-cell depleting antibody as a monotherapy,
iv) a combination therapy of an anti-Ab antibody and an anti-TNF biologic,
v) a combination therapy of an anti-Ab antibody and a B-cell depleting antibody, or
vi) a combination therapy of an anti-TNF biologic and a B-cell depleting antibody;
c) monitoring the subjects for changes in cognitive and functional outcomes over a predetermined trial period; and
d) comparing the efficacy and safety of the administered therapies across the unstratified AD population to identify effective treatment(s) for AD.

36. The method of claim 35, further comprising analyzing the trial data to identify one or more patient subsets based on genetic, inflammatory, and/or immune biomarker profiles that demonstrate better or worse treatment responsiveness to the therapies.

37. The method of claim 36, further comprising revising the product label for the at least one therapy, based on the identified patient subsets, to include specific indications, contraindications, dosage adjustments, or warnings directed to the patient subsets demonstrating better or worse treatment responsiveness.

38. A classical clinical trial in an unstratified population of AD subjects, the trial comprising:
a) enrolling subjects diagnosed with AD without prior stratification based on mechanism of action of therapies, mechanism of ARIA or CAA incidence;
b) randomly assigning the enrolled subjects to receive at least one therapy selected from the group consisting of:
i) an anti-Ab antibody as a monotherapy,
ii) an anti-TNF biologic as a monotherapy,
iii) a B-cell depleting antibody as a monotherapy,
iv) a combination therapy of an anti-Ab antibody and an anti-TNF biologic,
v) a combination therapy of an anti-Ab antibody and a B-cell depleting antibody, or
vi) a combination therapy of an anti-TNF biologic and a B-cell depleting antibody;
c) monitoring the subjects for changes in cognitive and functional outcomes over a predetermined trial period; and
d) comparing the efficacy and safety of the administered therapies across the unstratified AD population to identify effective treatment(s) for AD.

39. The clinical trial of claim 38, the trial further comprising a data analysis feature configured to analyze trial data to identify one or more patient subsets based on genetic and/or immune biomarker profiles who demonstrate better or worse treatment responsiveness to the therapies.

40. A method of administering a classical treatment protocol in an unstratified population of AD patients, comprising:
a identifying patients diagnosed with AD without prior stratification, wherein the stratification includes the mechanisms of action of therapies, the mechanism of ARIA or CAA incidence;
b) administering to the patients a therapy selected from the group consisting of:
i) an anti-Ab antibody as a monotherapy,
ii) an anti-TNF biologic as a monotherapy,
iii) a B-cell depleting antibody as a monotherapy,
iv) a combination therapy of an anti-Ab antibody and an anti-TNF biologic,
v) a combination therapy of an anti-Ab antibody and a B-cell depleting antibody, or
vi) a combination therapy of an anti-TNF biologic and a B-cell depleting antibody;
c) monitoring therapeutic outcomes and side effects in the treated patients over a predetermined treatment period; and
d) adjusting the therapy based on the observed efficacy and side effects in the patients, aiming to optimize therapeutic outcomes in the unstratified AD population.

41. The method of claim 40, further comprising analyzing the real-world evidence data to identify one or more patient subsets based on genetic, inflammatory, and/or immune biomarker profiles that demonstrate better or worse treatment responsiveness to the therapies.

42. The method of claim 41, further comprising revising the product label for the at least one therapy, based on the identified patient subsets, to include specific indications, contraindications, dosage adjustments, or warnings directed to the patient subsets demonstrating better or worse treatment responsiveness.

43. A pharmaceutical product label for treating AD in a patient, comprising:
a) instructions for administering a monotherapy or combination therapy comprising an anti-Ab antibody and at least one of an anti-TNF biologic or a B-cell depleting antibody;
b) indications for use based on therapeutic mechanisms, ARIA/CAA incidence mechanism, disease subtypes, the FcGR functional polymorphisms, and biomarker profiles to make treatment decisions;
c) guidelines for dose adjustments and administration frequency based on patient responsiveness and disease progression;
d) a warning section detailing potential side effects and contraindications specific to the therapies; and
e) a section on monitoring requirements for cognitive and functional status to assess therapy effectiveness.

44. A kit for stratifying AD patients for targeted therapy selection, comprising:
a) reagents for detecting ATN levels in a biological sample;
b) reagents for genotyping the FcGR functional polymorphisms in the patient;
c) reagents for measuring immune cells in peripheral blood;
d) reagents for measuring cytokines in peripheral blood;
e) instructions for interpreting the presence of ATN levels and FcGR functional polymorphisms to stratify patients into treatment groups;
f) guidelines for selecting appropriate AD therapies based on stratification; and
g) packaging material indicating the intended use of the kit for AD patient stratification and therapy selection.

45. A method of providing prior authorization for AD therapies, comprising:
a) receiving patient data, including the FcGR functional polymorphisms and ATN levels in a biological sample;
b) stratifying the patient into specific treatment groups based on the received patient data;
c) selecting an appropriate AD therapy for the patient from the group consisting of an anti-Ab antibody, an anti-TNF biologic, a B-cell depleting antibody, or a combination thereof, based on the stratification;
d) generating a prior authorization request that includes the selected therapy and justification based on the patient's stratification; and
e) transmitting the prior authorization request to a payer for approval, wherein the approval is based on the alignment of the selected therapy with the patient's stratification.

46. The method of claim 45, wherein the anti-Ab antibody is selected from the group consisting of lecanemab, donanemab, aducanumab, or gantenerumab.

47. The method of claim 45, wherein the anti-TNF biologic is selected from the group consisting of adalimumab, etanercept, or infliximab.

48. The method of claim 45, wherein the B-cell depleting antibody is an anti-CD20 antibody therapy, such as rituximab.

49. The method of claim 45, further including the step of adjusting the dosage and administration schedule of the selected therapy based on therapeutic mechanisms, ARIA/CAA incidence mechanism, disease subtypes, FcGR functional polymorphisms, and biomarker profiles to make treatment decisions.

50. The mechanism of claim 45, wherein the biological sample is selected from the group consisting of blood, plasma, and cerebrospinal fluid (CSF).

51. The method of claim 45, wherein the method is implemented using a computer system that processes patient data, stratifies patients, selects therapies, generates prior authorization requests, and communicates with payers.

52. The method of claim 45, wherein the payer is part of a health insurance network, government healthcare program, or a pharmacy benefit management (PBM).

53. The method of claim 45, further comprising following up with the payer to discuss alternative therapies or additional justifications if the initial prior authorization request is denied.

54. The method of claim 45, further comprising the step of monitoring the patient's progress on the selected therapy and submitting follow-up reports to the payer to support continued authorization.
